(19) United States
(12) Patent Application Publication
(10) Pub. No.: US 2006/0286141 A1
(43) Pub. Date: Dec. 21, 2006

(54) Systems for gel-based medical implants

(76) Inventor: Todd D. Campbell, (US)

Correspondence Address: KOLISCH HARTWELL, P.C., 200 PACIFIC BUILDING, 520 SW YAMHILL STREET, PORTLAND, OR, 97204, US

(21) Appl. No.: 10/587,580

(22) PCT Filed: Dec. 15, 2004
(86) PCT No.: PCT/US04/42474

§ 371 Date: Jul. 28, 2006

Related U.S. Application Data
(60) Provisional application No. 60/529,470, filed on Dec. 15, 2003.
(60) Provisional application No. 60/529,479, filed on Dec. 15, 2003.
(60) Provisional application No. 60/529,489, filed on Dec. 15, 2003.
(60) Provisional application No. 60/529,534, filed on Dec. 15, 2003.

(51) Int. Cl. A61K 9/14; A61F 2/06

(52) U.S. Cl. 424/423; 424/488; 623/1.11

(57) ABSTRACT

Systems, including methods and apparatus, for medical implants including a gel.

### CROSS-REFERENCES TO PRIORITY APPLICATIONS

This application is based upon and claims the benefit under 35 U.S.C. §119(e) of the following U.S. provisional patent applications: Ser. No. 60/529,470, filed Dec. 15, 2003; Ser. No. 60/529,479, filed Dec. 15, 2003; Ser. No. 60/529,489, filed Dec. 15, 2003; and Ser. No. 60/529,534, filed Dec. 15, 2003. Each of these applications is incorporated herein by reference in its entirety for all purposes.

### BACKGROUND

The human body has numerous vessels and organs that transport bodily fluids for nutrient delivery, recirculation and excretion of byproducts. Many of these structures have a tubular geometry, for example, blood vessels, the intestinal tract, and the bladder. Even relatively solid organs such as the heart, liver, kidney and pancreas have tubular cavities and lumens. Furthermore, disease processes such as tumors and aneurysms may create spaces or voids within otherwise solid organs.

The lumens afforded by organs and vessels can be affected by a variety of diseases and medical conditions. For example, a lumen may be occluded, thus limiting or blocking flow through the lumen. Since the lumen of many organs and vessels serve vital functions, such as providing a conduit for blood, urine, bile, or food, restriction of flow through the lumen is usually undesirable. The growth of an occluding atheroma in an artery is an exemplary restriction that impedes blood flow.

Devices, materials and methods for the treatment and repair of tissues around vessel or organ lumens continue to be developed to minimize or eliminate restrictions within the lumens. Many of the newer treatments access the medial, endomural zone of organs, organ components, or vessel tissues via surgical or percutaneous procedures. With many of these treatment procedures, inflammation, proliferative regrowth, and excessive ingrowth of tissue may occur in response to the trauma or vascular damage near the treatment area, lessening clinical effectiveness.

Medical researchers of coronary disease, for example, are working to develop better medical practices for inhibiting stenosis, the narrowing or constricting of a blood vessel lumen, and for preventing or minimizing restenosis that may occur after a procedure such as angioplasty. Atherosclerosis, which is characterized by the progressive buildup of hard plaque in the coronary arteries, as well as other types of stenoses are treated by a number of procedures, including balloon dilatation, stenting, ablation, atherectomy or laser treatment. Stenosis, restenosis, and cancerous growth or tumors may block other body passageways besides coronary arteries, including the esophagus, bile ducts, trachea, intestine, and the urethra, among others.

Although angioplasty and stenting procedures are probably the best-known procedures for treating stenosis within vessels, other treatments are available. In cases of severe atherosclerotic obstructions, endovascular removal of obstructive lesions via endovascular atherectomy, a catheter-based cutting or drilling procedure from within the vessel, may be employed. For example, directional coronary atherectomy involves a small sharp blade directed from inside a catheter to cut and ablate plaque from the wall of the artery. For another example, rotational atherectomy or rotablation procedures drill through plaque with a diamond-coated burr and pulverize the buildup of cholesterol or other fatty substances into small particles that can enter the bloodstream. While these procedures remove the diseased atheroma close to the vessel lumen and treatment device, they do not address the source or core of the disease that often lies in the vessel media.

One common minimally invasive medical procedure for treating various coronary artery diseases is percutaneous transluminal coronary angioplasty (PTCA), also called balloon angioplasty. PTCA can relieve myocardial ischemia by reducing lumen obstruction and improving coronary flow. After a catheter is introduced into a blood vessel and advanced to a treatment site, a small dilating balloon at the distal end of the catheter is passed across an atherosclerotic plaque and inflated to compress the plaque and expand an occluded region of the blood vessel. This compression cracks or otherwise mechanically deforms the lesion and increases the lumen size of the vessel, which in turn increases blood flow. In PTCA, the blockage is not actually removed, but is compressed into the arterial walls.

While PTCA represents therapeutic advances in the treatment of coronary artery disease, vessel renarrowing or reclosure of the vessel often occurs after PTCA, due in part to trauma of the vessel caused by the balloon dilation or stent placement. In some cases, the vessel reverts either abruptly or progressively to its occluded condition, limiting the effectiveness of the PTCA procedure.

A medical implant such as an intravascular stent may be used to support the vessel, thus mechanically keeping the vessel open and preventing post-angioplasty vessel reclosure. One common catheter procedure delivers the stent in a compressed form to the treatment site where the stent expands via the inflation of a catheter balloon or through self-expansion to engage the wall of the coronary or peripheral vessel. Most stents are fabricated from metals, alloys or polymers and remain in the blood vessel indefinitely. Stent manufacturers have developed stents of various diameters and lengths to allow anatomic flexibility, although the stents may not be flexible enough to conform completely to the shape of the vessel being treated. In some cases, a stent itself can cause undesirable local thrombosis, create restenosis due to over-expansion within the vessel, or result in metal ion migration from the stent latticework.

Restenosis, the gradual narrowing of a vessel, can occur after interventional procedures such as stenting and angioplasty that traumatize the vessel wall. Such trauma may lead to the formation of local thrombosis or blood clotting, which is most likely to occur soon after an intravascular procedure. To address the problem of thrombosis, patients receiving stents also may receive extensive systemic treatment with anti-coagulants such as aspirin and anti-platelet drugs.

An uncontrolled migration and proliferation of smooth muscle cells, combined with extracellular matrix production, may develop during the first three to six months after a procedure when vessel trauma occurred. Scar-like proliferation of endothelial cells that normally line blood vessels may incur restenosis, and with stent placement, there may be an ingrowth of tissue proliferation or inflammatory material through the interstices of the stent that can block and occlude the vessel.

Unfortunately, restenosis frequently necessitates further interventions such as repeat angioplasty or coronary bypass surgery. Alternative procedures, such as delivering radiation with intracoronary brachytherapy, have been used in an effort to curtail overproduction of cells in the traumatized area.

A significant amount of medical research continues to focus on the prevention and treatment of hard and soft plaque within vessels, one area of study being local drug delivery to diseased or traumatized treatment areas. For example, in an effort to prevent restenosis provoked by medical procedures, systems and methods have been developed to locally deliver pharmacological agents such as rapamycin, an immunosuppressant known for its anti-proliferation properties, or paclitaxel, a chemotherapy agent and microtubular stabilizer that causes cells to stop dividing due to a mitotic block between metaphase and anaphase of cell division. Some of these inhibitory pharmacological agents have the potential to interfere or delay healing, weakening the structure or elasticity of the newly healed vessel wall and damaging surrounding endothelium and/or other medial smooth muscle cells. Dead and dying cells release mitogenic agents that may stimulate additional smooth muscle cell proliferation and exacerbate stenosis.

While restenosis from hard-plaque obstructions can be a cause of myocardial infarction, known commonly as a heart attack, recent medical research suggests that the development and rupture of non-occlusive, soft atherosclerotic or vulnerable plaques in coronary arteries may play a greater role in heart attacks than restenosis caused from hard plaques. Research suggests that vulnerable plaques have a dense infiltrate of macrophages within a thin fibrous cap that overlies a pool of lipid. Vulnerable plaque is formed from droplets of lipid that are absorbed by an artery, which can cause the release of proteins called cytokines that exacerbate inflammation. The cytokines act as an adhesive, attracting monocytes, so-called immune-system cells, to the artery wall where they push into the tissue of the wall. The monocytes change into macrophages, cells of the reticuloendothelial system, which begin to soak up fat droplets and form a plaque with a thin covering.

The rupture of vulnerable plaques, due to inflammatory processes and mechanical stress like increased blood pressure, results in exposure of blood to the lipid core and other plaque components. Vulnerable plaque erodes or ruptures, creating a raw tissue surface that forms scabs, and pieces of plaque that break off may accumulate in the coronary artery to create a thrombus of sufficient size to slow down or stop blood flow.

Vulnerable plaque is ingrained under the arterial wall and is difficult to detect with conventional means such as angiography or fluoroscopy. Thermography, which is capable of detecting a temperature difference between atherosclerotic plaque and healthy vessel walls, is one of the imaging methods being pursued for locating vulnerable plaque.

Unnecessary tissue damage continues to be an issue for many percutaneous procedures and endoluminal treatments of diseased vessels. Therefore, improved systems, including methods and apparatus, for treating diseased organ lumens, blood vessels, and other endoluminal vessels are needed to minimize or eliminate damage to surrounding tissue, to prevent restenosis of treated areas, and/or to prevent inflammation of diseased areas. The desirable treatment of specific tissues may provide mechanical support for the lumen and sustained local delivery of therapeutic compositions to help tissue to heal while avoiding excessive drug levels. More specifically, improved systems for treating coronary artery disease may minimize inflammation, restenosis, and/or the ingrowth of host tissue proliferation; control the dosage and delivery of therapeutic components to vascular tissue and smooth muscle cells over extended periods of time; successfully treat vulnerable plaque; and/or treat or prevent undesirable medical conditions within a vessel.

### SUMMARY

The present teachings provide systems, including methods and apparatus, for medical implants including a gel.

### BRIEF DESCRIPTION OF THE DRAWINGS

Various embodiments of the present teachings are illustrated by the accompanying figures, with the figures not necessarily drawn to scale.

FIG. 1 is a side view of an exemplary coated stent, in accordance with aspects of the present teachings.

FIG. 2 is a sectional view of the coated stent of FIG. 1, taken generally along line A-A′ of FIG. 1.

FIG. 3 is a partially sectional view of the coated stent of FIG. 1 deployed in a vessel and releasing therapeutic agents, in accordance with aspects of the present teachings.

FIG. 4 is a schematic diagram of an exemplary method of coating a medical implant with a gel, in accordance with aspects of the present teachings.

FIG. 5 is a flow diagram of an exemplary method of treating a vessel in a mammalian body, in accordance with aspects of the present teachings.

FIG. 6 is a view of an exemplary system for treating a vessel in a mammalian body, in accordance with aspects of the present teachings.

FIG. 7 is a longitudinal sectional view of an exemplary gel-based stent, in accordance with aspects of the present teachings.

FIG. 8 is a sectional view of the gel-based stent of FIG. 7, taken generally along line A-A′ of FIG. 7.

FIG. 9 is a view of an exemplary gel-based stent formed to include a plurality of apertures, in accordance with aspects of the present teachings.

FIG. 10 is a flow diagram of an exemplary method of treating a vessel in a mammalian body, in accordance with aspects of the present teachings.

FIG. 11 is a longitudinal sectional view of an exemplary alginate stent being formed within a vessel of a mammalian body, in accordance with aspects of the present teachings.

FIG. 12 is a longitudinal sectional view of an exemplary alginate stent formed within a vessel of a mammalian body, in accordance with aspects of the present teachings.

FIG. 13 is a flow diagram of an exemplary method of forming an alginate stent in a vessel of a mammalian body, in accordance with aspects of the present teachings.

FIG. 14 is a longitudinal sectional view of an exemplary alginate stent being formed within a vessel of a mammalian body, in accordance with aspects of the present teachings.

FIG. 15 is a longitudinal sectional view of an exemplary alginate stent formed within a vessel of a mammalian body, in accordance with aspects of the present teachings.

FIG. 16 is a flow diagram of another exemplary method of forming an alginate stent in a vessel of a mammalian body, in accordance with aspects of the present teachings.

FIGS. 17a-f are longitudinal sectional views of exemplary configurations produced by performing an exemplary method of forming an alginate stent in a mammalian body, in accordance with aspects of the present teachings.

FIG. 18 is a longitudinal sectional view of an exemplary alginate stent formed within a vessel of a mammalian body, in accordance with aspects of the present teachings.

FIG. 19 is a flow diagram of yet another exemplary method of forming an alginate stent in a vessel of a mammalian body, in accordance with aspects of the present teachings.

FIG. 20 is a view of an exemplary alginate bioreactor for treating a mammalian body, in accordance with aspects of the present teachings.

FIG. 21 is a somewhat schematic view of an exemplary system for forming an alginate bioreactor in a mammalian body, in accordance with aspects of the present teachings.

### DETAILED DESCRIPTION

The present teachings provide systems, including methods and apparatus, for medical implants including a gel. The implants may be formed outside of implant recipients or in situ in the recipients. The gel may be a coating disposed on the body of each implant and/or may form at least a substantial portion of the body of each implant. The gel may include source components configured to serve as a source of therapeutic agents released from the gel. The source components may include therapeutic components (chemical substances) and/or cellular components (cells). The implants may be used to treat and/or prevent any suitable medical condition, such as vulnerable plaque, stenosis, restenosis, thrombosis, saphenous vein graft disease, and/or diabetes. Other medical conditions that may be treated and/or prevented with the implants are disclosed elsewhere in the present teachings.

The present teachings may provide a stent including a stent latticework and a gel-based coating (such as an alginate gel) disposed on the stent latticework.

The present teachings may provide a method of treating a vessel in a mammalian body. The method may include steps of providing a stent latticework and coating the stent latticework with an alginate solution to form a coated stent having an alginate coating disposed on the stent latticework. The coated stent may be positioned within the vessel and deployed (for example, allowed to expand into engagement with the vessel wall from a compressed state). A therapeutic agent may be eluted from the alginate coating.

The present teachings may provide an alginate coating for an implantable medical device. The alginate coating may include an alginate matrix and one or more therapeutic components and/or one or more cellular components (cells) (and/or types of cellular components) dispersed within the alginate matrix.

The present teachings may provide a gel-based implant, such as an alginate stent and/or cap (lining) for treating a vessel in a mammalian body. The implant may provide support, release therapeutic agents, and/or the like. In some examples, the implant may be configured to cover vulnerable plaque. The implant may include an alginate matrix in contact with an endoluminal wall of the vessel and/or vulnerable plaque and a central lumen extending axially through the alginate matrix.

The present teachings may provide a method of treating a vessel and/or vulnerable plaque in a mammalian body. A gel-based implant (such as an alginate stent and/or a cap) may be formed within the vessel, and a therapeutic agent may be eluted from one or more therapeutic components and/or cellular components (cells) dispersed within the implant. The implant may be in contact with an endoluminal wall of the vessel (and/or vulnerable plaque thereof) and may have a central lumen extending axially through the alginate stent.

The present teachings may provide a system for forming a gel-based implant (such as an alginate stent and/or cap) in a mammalian body. The system may include an implant formation catheter having a catheter body, a formation balloon attached to the catheter body near a distal end of the catheter body, and a gel-delivery lumen within the catheter body. An implant may be formed on an endoluminal wall of the vessel (and/or on vulnerable plaque) from a fluent pre-gel solution (such as an alginate solution) injected through the gel-delivery lumen into a cavity between the formation balloon and an endoluminal wall of the vessel when the formation balloon is inflated.

The present teachings may provide a method of forming a gel-based implant (such as an alginate stent and/or a cap) in situ in a vessel of a mammalian body. An implant-formation catheter having a catheter body may be positioned in the vessel. A formation balloon may be attached to the catheter body near a distal end of the catheter body and may be inflated. A pre-gel solution (such as a fluent alginate solution) may be injected through a gel-delivery lumen into a cavity formed between the inflated formation balloon and an endoluminal wall of the vessel. The pre-gel solution may harden (gel such as by cross-linking) from a fluent to a nonfluent state to form the implant.

The present teachings may provide a system for forming a gel-based implant (such as an alginate stent and/or cap) in a mammalian body, for example, to treat vulnerable plaque and/or reduce stenosis, among others. The system may include an implant-formation catheter having a catheter body. A distal occlusion balloon may be attached to the catheter body near a distal end of the catheter body. A proximal occlusion balloon may be attached to the catheter body proximal to the distal occlusion balloon. A medial formation balloon may be attached to the catheter body between the distal occlusion balloon and the proximal occlusion balloon. A gel-delivery lumen may be included within the catheter body. A gel-based implant may be formed from a pre-gel solution (such as a fluent alginate solution) injected through the gel-delivery lumen into a cavity between the medial formation balloon and an endoluminal wall of the vessel when the distal occlusion balloon and the proximal occlusion balloon are inflated.

The present teachings may provide a method of forming a gel-based implant (such as an alginate stent and/or cap, among others) in a vessel of a mammalian body. An implant-formation catheter having a catheter body may be positioned in the vessel. A distal occlusion balloon may be attached to the catheter body near a distal end of the catheter body and may be inflated. A proximal occlusion balloon may be attached to the catheter body proximal to the distal balloon and may be inflated. A medial formation balloon may be attached to the catheter body between the distal occlusion balloon and the proximal occlusion balloon and may be inflated. A pre-gel solution may be injected through a gel-delivery lumen into a cavity formed between the inflated distal occlusion balloon, the inflated proximal occlusion balloon, the inflated medial formation balloon, and an endoluminal wall of the vessel. The pre-gel solution may be hardened (gelled) from a fluent to a nonfluent state to form the implant, for example, to cover vulnerable plaque and/or to create an endoluminal lining and/or support, among others.

The present teachings may provide a system for forming a gel-based implant (such as an alginate stent and/or cap, among others) in a mammalian body. The system may include an implant-formation catheter having a catheter body, an angioplasty balloon attached to the catheter body near a distal end of the catheter body, a formation balloon attached to the catheter body proximal to the angioplasty balloon, and a gel-delivery lumen within the catheter body. A gelling agent, configured to stimulate formation of a gel from a pre-gel solution, may be disposed on a surface of the angioplasty balloon. A gel-based implant may be formed from a pre-gel solution injected through the gel-delivery lumen into a cavity between the formation balloon and an endoluminal wall of the vessel when the formation balloon is inflated.

The present teachings may provide a method of forming a gel-based implant (such as an alginate stent, cap, and/or lining, among others) in a vessel of a mammalian body, for example on vulnerable plaque and/or an endoluminal wall of the vessel. An implant-formation catheter having a catheter body may be positioned at a first location in the vessel. An angioplasty balloon may be attached to the catheter body near a distal end of the catheter and may include a gelling (or linking) agent configured to stimulate formation of a gel from a pre-gel solution, disposed on a surface of the angioplasty balloon, and may be inflated. The gelling agent may be deposited on an endoluminal wall of the vessel. The angioplasty balloon may be deflated and repositioned at a second location in the vessel distal to the first location. The angioplasty balloon may be re-inflated. A formation balloon may be attached to the catheter body proximal to the angioplasty balloon and inflated. A fluent pre-gel solution may be injected through a gel-delivery lumen into a cavity formed between the formation balloon and an endoluminal wall of the vessel. The pre-gel solution may be hardened (gelled) by the gelling agent deposited on the endoluminal wall of the vessel.

The present teachings may provide a system for forming a gel-based implant (such as a stent, cap, and/or lining, among others) in a vessel of a mammalian body. The system may include an implant-formation catheter having a catheter body and a gel-delivery lumen within the catheter body, and at least one formation balloon attached proximal to a distal end of the catheter body. A gel-based implant may be formed in the vessel when the implant-formation catheter is inserted into the vessel and a pre-gel solution is injected through the gel-delivery lumen into a cavity formed between the formation balloon and an endoluminal wall of the vessel.

The present teachings may provide a method of forming a gel-based implant in a vessel of a mammalian body. An implant-formation catheter with at least one formation balloon may be inserted into the vessel. A pre-gel solution may be injected into a cavity formed between the formation balloon and an endoluminal wall of the vessel when the formation balloon is inflated. The pre-gel solution may be hardened (gelled) to form the implant, and the implant-formation catheter may be withdrawn from the vessel. The implant thus formed may be in contact with the endoluminal wall of the vessel and may include a central lumen extending axially through the implant.

The present teachings may provide a gel-based (such as alginate) bioreactor for treating a mammalian body. The bioreactor may include a gel matrix and a therapeutic component and/or a cellular component dispersed within the gel matrix. The therapeutic and/or cellular component may be configured so that a therapeutic agent is released from the gel matrix after the bioreactor is formed within a mammalian body.

The present teachings may provide a method of treating a medical condition in a mammalian body. A gel-based (such as alginate) bioreactor including a gel matrix may be formed within a portion of the mammalian body. The bioreactor may include a chemical substance(s) and/or cells dispersed within the bioreactor and may be configured to serve as a source of a therapeutic agent that is released progressively from the bioreactor.

Another aspect of the invention is a system for forming an alginate bioreactor in mammalian body. The system may include a first chamber, a second chamber, and an alginate solution injector fluidly coupled to the first chamber and the second chamber. An alginate solution from the first chamber may be injected into a portion of the mammalian body with an alginate linking and/or gelling agent from the second chamber to form the alginate bioreactor.

I. Gels

The implants of the present teachings may include and/or may be formed at least substantially of a gel. A gel, as used herein, is any semi-solid material formed by a solid matrix holding liquid. The gel may be bioabsorbable (bio-erodible) or nonbioabsorbable. Exemplary gels may include a matrix formed of protein, polysaccharides, synthetic compounds, etc. In some examples, the gels may be hydrogels, that is, gels including water substantially or completely as the liquid. The matrix of a gel may be formed partially or completely of any suitable material, for example, alginate, karaya gum, gelatin, albumin, collagen, polymalic acid, polyamino acids, polyacrylates, polyethylene glycols, starch, cellulose, guar gum, agar, carrageenans, pectin, polyglycolides, polylactides, polydioxanones, and/or the like. The matrix may include one or more types of subunits that are polymerized and/or cross-linked into a network to form the matrix.

The gel may include a matrix formed at least substantially of alginate. An alginate matrix generally includes a three-dimensional matrix formed at least substantially of guluronate and/or mannuronate subunits, and/or derivatives thereof. Alginate may be obtained from any suitable source, for example, extracted from brown seaweeds, such as Phaeophyceae and Laminaria. In addition, the alginate may be isolated in, or processed into, a fluent (pre-gel) form of linear chains including guluronate and/or mannuronate subunits. The chains may be cross-linked with a gelling (linking) agent, to produce a substantially nonfluent three-dimensional matrix. Each chain may include any ratio of guluronate and/or mannuronate subunits, and in any relative disposition. In some examples, each chain may include homopolymeric blocks of mannuronate alginate subunits and/or guluronate alginate subunits, covalently linked together in different sequences or blocks. In some examples, the alginate subunits can appear in homopolymeric blocks of consecutive guluronate alginate subunits, consecutive mannuronate alginate subunits, alternating mannuronate alginate subunits and guluronate alginate subunits, other systematic arrangements, or randomly organized blocks. The relative amount of each block type may vary with the source of the alginate. Alternating blocks of mannuronate alginate subunits and guluronate alginate subunits may form more flexible chains and may be more soluble at lower pH than other block configurations. Blocks of guluronate alginate subunits may form stiffer chain elements, and two guluronate alginate subunitic blocks of more than six subunits each may form stable cross-linked junctions with divalent cations such as Ca²⁺, Ba²⁺, Sr²⁺, and Mg²⁺, leading to a three-dimensional gel network or alginate matrix.

At low pH, protonized alginates may form acidic gels. The homopolymeric blocks may form the majority of the junctions, and the relative content of guluronate alginate subunits may determine the stability of the gel.

In some examples, alginate gels can develop and set at temperatures close to room temperature. This property may be useful in applications involving fragile materials like cells or tissue with low tolerance for higher temperatures.

The alginate polymers may serve as thermally stable cold-setting gels in the presence of divalent cations, such as calcium ions from calcium sources. Gelling can depend on ion binding, with divalent cation addition being important for the production of homogeneous gels, for example, by ionic diffusion or controlled acidification of calcium carbonate. High guluronate alginate subunit content may produce strong, brittle gels with good heat stability, whereas high mannuronate alginate subunit content may produce weaker, more elastic gels. At low or very high divalent calcium concentrations, high mannuronate alginates may produce stronger gels. When the average chain lengths are not particularly short, gelling properties may correlate with the average guluronate alginate subunit block length having an optimum block size of about twelve subunits, and do not necessarily correlate with the ratio of mannuronate alginate subunits to guluronate alginate subunits, which may be due primarily to alternating mannuronate-guluronate chains. Recombinant epimerases with different specificities may be used to tailor mechanical and transport characteristics of the alginate.

The solubility and water-holding capacity of the alginate may depend at least on pH, molecular weight, ionic strength, and the nature of the ions present. Alginate tends to precipitate below a pH of about 3.5. Alginate with lower molecular weight calcium alginate chains of less than 500 subunits shows increasing water binding with increasing size. Lower ionic strength of alginate increases the extended nature of the calcium alginate chains. An alginate gel may develop rapidly in the presence of divalent cations like Ca²⁺, Ba²⁺, Sr²⁺, or Mg²⁺ and acid gels may also develop at low pH. Gelling of the alginate premix may occur when divalent cations take part in the interchain ionic binding between guluronate alginate subunit blocks in the polymer chain, giving rise to a three-dimensional network. Alginates with a high content of guluronate alginate subunit blocks may tend to produce stronger gels. Gels made of mannuronate-rich alginate may be softer and more fragile, with a lower porosity, due in part, for example, to a lower binding strength between the polymer chains and to a greater flexibility of the molecules. An alginate gel (a gel coating and/or a gel-based implant body of an implant) may include a matrix of mannuronate alginate subunits and guluronate alginate subunits in a predetermined ratio within cross-linked chains to provide the desired mechanical strength and flexibility while controlling the elution rates for therapeutic agents (see Section II).

The gelling process may be highly dependent on diffusion of gelling ions into the polymer network. Methods that may be used for the preparation of alginate gels may include dialysis/diffusion and internal gelling.

In the dialysis/diffusion or diffusion-setting method, gelling ions may be allowed to diffuse into the alginate solution. This method may be used for immobilization of living cells in the alginate gel. An alginate solution can also be solidified by internal gelation, internal setting, or in situ gelling. A calcium salt with limited solubility or complexed divalent calcium ions may be mixed into an alginate solution, resulting in the release of calcium ions, usually by the generation of acidic pH with a slowly acting acid such as D-glucono-α-lactone. The resultant alginate may be a homogeneous alginate macrogel. Diffusion setting and internal setting of the alginate matrix may have different gelling kinetics and may result in differences in gel networks.

II. Therapeutic Agents

Implants of the present teachings may be configured to serve as a source of therapeutic agents released from a gel of the implants. The therapeutic agents may be released from the gel with any suitable kinetics over any suitable time period, such as minutes, hours, days, weeks, months, years, etc. Release of the agents may occur through diffusion from the gel, fluid flow through the gel, breakdown of the implants (such as by bio-erosion of the gel and particularly a matrix thereof), lysis of cells in the matrix, secretion from cells in the matrix, and/or the like.

The therapeutic agents may have any suitable relationship to source components included in the gels. The therapeutic agents may be structurally identical to the source components, chemical derivatives of the source components (such as derivatives produced by cleavage, oxidation, reduction, addition, cyclization, isomerization, and/or removal of moieties from the source components), and/or products of the source components (such as metabolites of cells).

The source components may be introduced into gels by any suitable approach. For example, the source components may be (1) present during, and trapped/encapsulated by, gel matrices during their formation, and/or (2) introduced after matrix formation (such as by diffusion by soaking the gels in solutions containing the source components). The source components thus may be retained noncovalently or covalently by gel matrices. Covalently retained source components may be bonded to gel matrices before, during, and/or after formation of the matrices. For example, the source components may be bonded to polymer chains before and/or after the chains are cross-linked to form gel matrices.

One or more source components disposed in a gel may be therapeutic components (chemical substances) and/or cellular components (cells) that act as a source of one or more therapeutic agents. Therapeutic agents released from a gel coating and/or a gel implant body may include, for example, nitric oxide, vascular endothelial growth factor, a biological anti-inflammatory agent, vitamin C, acetylsalicylic acid, a lipid lowering compound, a high-density lipoprotein cholesterol, a streptokinase, a kinase, a thrombolytic agent, an anti-thrombotic agent, a blood-thinning agent, a coumadin material, an anti-cancer agent, an angiogenic agent, an anti-angiogenic agent, an anti-rejection agent, a hormone, a therapeutic component, a cellular component, or a combination thereof.

A. Therapeutic Components

In some embodiments, therapeutic components may be dispersed within a gel coating and/or gel-based implant body of an implant. Therapeutic components within a gel coating or implant body may be any suitable substance, including, for example, an anti-coagulant, an anti-platelet drug, an anti-thrombotic drug, an anti-proliferant, an inhibitory agent, an anti-stenotic substance, heparin, a heparin peptide, an anti-cancer drug, an anti-inflammatant, nitroglycerin, L-arginine, an amino acid, a nutraceutical, an enzyme, a nitric oxide synthase, a diazeniumdiolate, matrix metalloproteinase, a nitric oxide donor, rapamycin, a rapamycin analog, paclitaxel, a paclitaxel analog, a coumadin therapy, a lipase, or a combination thereof. Therapeutic agents released from a gel having therapeutic components may include, for example, the components themselves and/or derivatives thereof.

Since it is such a small molecule, nitric oxide can diffuse rapidly across cell membranes and, depending on the conditions, is able to diffuse distances of more than several hundred microns, as is demonstrated by its regulation of smooth muscle cells, vascular dilation, tissue compliance and physiological tone of the vessel. Nitric oxide may be produced within a gel matrix, such as an alginate matrix configured as a gel coating and/or gel-based implant body, and then delivered directly to a vessel. For example, L-arginine, a naturally occurring amino acid, and/or other nutraceuticals may be converted to nitric oxide within an alginate or other gel matrix by a group of enzymes such as nitric oxide synthases. These enzymes convert L-arginine into citrulline, producing nitric oxide in the process. In another example, nitric oxide is liberated from diazeniumdiolates, compounds that release nitric oxide into the blood stream and vascular walls.

B. Cellular Components

Cellular components within a gel coating and/or gel-based implant body may include any suitable living or dead cells of one or more types. The cells may be, for example, endothelial cells, manipulated cells of designer deoxyribonucleic acid, host-derived cells from a host source (that is, from the intended recipient of a stent or other implant), donor-derived cells from a donor source (other than the recipient), pharmacologically viable cells, freeze-dried cells, or a combination thereof. Therapeutic agents released from a gel having cellular components may include, for example, a residue, a byproduct, and/or natural secretion from the cells.

In some examples, the cellular components may include endothelial cells that produce nitric oxide, a regulating molecule for smooth muscle cell quiescence and maintenance of vascular smooth muscle cells in the non-proliferative stage. A patient's own endothelial cells from, for example, microvascular adipose tissue may be harvested and mixed with a pre-gel solution (such as a fluent alginate solution). The cells then may be encapsulated in the gel produced by gelation. This gel may be formed on a medical implant (such as a coating on a stent) and/or may form at least a substantial portion of the implant. Upon implantation, the cells may remain viable (living) and locally may produce nitric oxide to regulate and maintain the quiescent nature of smooth muscle cells, which can be a contributor to the production and recruitment of fibroblasts from the media and adventitia of arteries. With the continued long-term production of nitric oxide from translocated endothelial cells, vascular patency may be maintained for a period substantially longer than the period for potential stenotic reoccurrence following stent placement.

In some examples, cells, such as endothelial cells, from either a host or donor source may be preserved with trehalose and freeze-dried, rendering the cells functional yet in a dehydrated state. The cells may be mixed into an alginate solution (or other pre-gel solution) and then used to coat an implant and/or form the implant body of the implant. Use of cells in a preserved fashion may allow for manufacturing of an implant in advance of a medical procedure. The cells may be preserved with trehalose and protected by the immune barrier of the alginate or other gel matrix. One skilled in the art can identify alternative cell-producing components that can be substituted for endothelial cells and provide therapeutic agents from a gel matrix.

In the case of cellular components, a gel matrix (such as an alginate matrix) may serve as an immune barrier so that the immune system of the recipient does not recognize cellular components contained within the gel matrix. Accordingly, the immune system may be restricted from killing and/or destroying the cells and thus terminating the production of therapeutic agents by the cells. In addition, the gel matrix may allow for the passage of nutrients, wastes, and therapeutic proteins and agents through the gel matrix into the surrounding vessel (and/or from the vessel into the matrix). Therapeutic agents thus may be delivered in close proximity to the treatment site. With imbibed cellular and therapeutic components, long-term release of therapeutic agents from the gel coating may be provided.

Living cells or other biomaterials and therapeutic compounds may be immobilized in an alginate matrix. Cells immobilized in alginate gels may maintain good viability during long-term culture, due in part to the mild environment of the gel network. An alginate gel may provide a physically protective barrier for immobilized cells and tissue, and may inhibit immunological reactions of the host.

An alginate matrix may provide a location that is viable and productive for cellular components. This viable and productive location may be possible because an alginate matrix allows diffusion of nutrients to cells, diffusion of respiratory byproducts to the surrounding area, and diffusion of selected therapeutic components in an unaltered condition from the alginate matrix. In some cases, an alginate matrix may serve as an immune barrier while providing for diffusive transport for therapeutic and cellular materials. The immune barrier properties of an alginate matrix may be particularly useful for non-host derived cell sources, or manipulated cells of designer deoxyribonucleic acid (DNA).

III. Gel-Coated Implants

Implants of the present teachings may include an implant body and a gel coating disposed partially or at least substantially completely over the surface of the body.

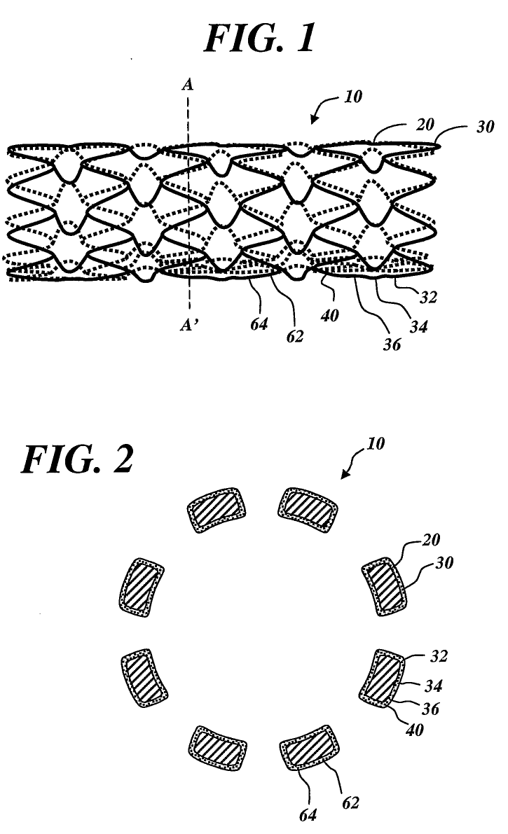

FIG. 1 illustrates an example of a coated implant, such as a coated stent, constructed in accordance with aspects of the present teachings. FIG. 2 illustrates a cross-sectional view of the coated implant of FIG. 1, with like-numbered elements referring to similar or identical elements in each illustration. Coated stent 10 may include a stent latticework 20 with an alginate or other gel-based coating 30 disposed on stent latticework 20. Alginate coating 30 may provide a protective coating for stent latticework 20 to minimize, for example, emission of metal ions. Alginate coating 30 also may provide a mechanism for controlled, time-release characteristics of therapeutic agents 40 from any therapeutic components 34 and cellular components (cells) 36 disposed within an alginate matrix 32 of alginate coating 30. In some examples, the present teachings may provide localized delivery of one or more therapeutic agents 40 from therapeutic components 34 dispersed within alginate coating 30 when coated stent 10 is deployed (positioned and/or expanded) within a lumen of a mammalian recipient. In some examples, the present teachings may provide long-term delivery of one or more therapeutic agents 40 via a matrix suitable for encapsulating living (and/or dead) cells from transplanted or implanted cells that produce such therapeutic agents.

Stent latticework 20 or other implantable medical devices may be covered with a relatively thin coating of alginate matrix 32 including selected therapeutic components 34 and cellular components 36 that produce therapeutic agents 40 for elution from alginate coating 30. The alginate coating thus may serve as a source of these therapeutic agents.

Stent latticework 20 of coated stent 10 may comprise, for example, a metallic body or a polymeric body. Metallic bodies generally may be formed of any biocompatible metal or metal alloy, including stainless steel, nitinol, platinum, and/or titanium, among others. Polymeric bodies may include, for example, a non-absorbable polymer, such as polyethylene, and/or a bio-absorbable polymer such as poly-lactide, poly-galactide, lactide/galactide co-polymers, polydioxanones, and/or other bio-erodable polymers suitable for implantation within a mammalian (such as human) body.

Stent latticework 20 of coated stent 10 may be, for example, balloon-expandable or self-expandable, which are stent configurations that are well known in the art. Balloon-expandable stents may be crimped onto an inflatable polyurethane balloon that is coupled near a distal end of a catheter body. Inflation lumens within the catheter body may allow an inflation fluid to be transported into and out from an interior region of the inflatable balloon. When coated stent 10 is appropriately positioned within the vessel, the stent may be expanded by inflating the balloon, thereby enlarging stent latticework 20 and deforming the latticework against the endoluminal wall of the vessel to provide mechanical support and allow for elution of one or more therapeutic agents 40 from alginate coating 30.

Alternatively, a self-expandable stent latticework 20 may expand and press against endoluminal walls of the vessel, for example, when a compression retainer such as a deployment sheath is pulled away from the stent latticework so that the compressed stent latticework freely expands towards its original expanded shape.

Gel coating 30 may include an alginate matrix 32, and may include one or more therapeutic components 34 and/or cellular components 36. Gel coating 30 may be configured to control the elution (release) of one or more therapeutic agents 40 from either therapeutic components 34 or cellular components 36 in the coating.

In some embodiments, coated stent 10 may include one or more cellular components 36 dispersed within alginate coating 30. Cellular components 36 and the gel matrix may be configured so that therapeutic agent 40 is released when coated stent 10 is deployed within a vessel of a mammalian body, for example by diffusion and/or cleavage of chemical bonds, among others.

In some examples, long-term administration of at least one therapeutic agent 40 such as nitric oxide may be provided by an implant to a mammalian vessel. Endothelial-derived nitric oxide is a naturally occurring regulation compound. The endothelial cell lining of vessels produces the nitric oxide molecule. Endogenously produced nitric oxide is produced by the endothelial cell in such a manner that the uptake of the molecule regulates the proliferation of the vascular smooth muscle cells and maintains the cellular quiescence of smooth muscle cells within the vascular architecture. Nitric oxide may be critical to numerous biological processes, including vasodilation, neurotransmission, and macrophage-mediated microorganism and tumor killing. Nitric oxide may be administrated in a chemically synthesized form as a nitric oxide donor, such as nitroglycerin dispersed within alginate matrix 32.

Disruption of the endothelial lining in the vessel may result in the reduction of nitric oxide production, leading to the loss of regulation of the smooth muscle cells. This disruption can occur during stent placement, angioplasty, or from disease accumulation. Stent placement and angioplasty procedures that open an occluded vessel exert significant pressure on the luminal surface and may damage the endothelial cells.

FIG. 2 illustrates a sectional view of the coated stent of FIG. 1, taken through line A-A′. Coated stent 10 may include a stent latticework 20 and an alginate coating 30 disposed on stent latticework 20. Since alginate coating 30 may be thin relative to the spacing between struts of stent latticework 20. Alginate coating 30 may individually coat the struts and/or other members of stent latticework 20.

Alginate coating 30 may include an alginate matrix 32 with one or more therapeutic components 34 or cellular components 36 dispersed within alginate coating 30. For example, therapeutic components 34 and cellular components 36 can be either uniformly dispersed throughout alginate coating 30, or have a non-uniform profile with a higher concentration of therapeutic components 34 or cellular components 36 nearer the struts of stent. latticework 20 or closer to an outer surface of alginate coating 30. In another example, therapeutic components 34 and cellular components may agglomerate or collect in regions of alginate coating 30.

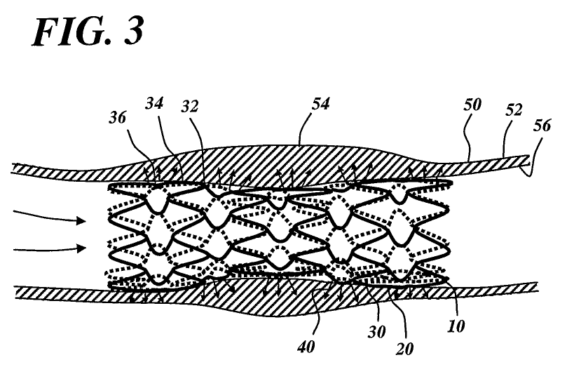

FIG. 3 illustrates a coated stent deployed in a vessel, in accordance with aspects of the present teachings. In either a balloon-expandable or self-expanding configuration, a coated stent 10 with a stent latticework 20 and an alginate coating 30 may be deployed in a vessel 50 of a mammalian body 52. Vessel 50 may have a partial occlusion or stenosed region 54 that blocks the flow of fluid through vessel 50. With coated stent 10 deployed in stenosed region 54, endoluminal walls 56 may be locally expanded outward to reduce the constriction and allow for increased fluid flow through the vessel.

Alginate coating 30 includes an alginate matrix 32 and one or more therapeutic components 34 or cellular components 36. Therapeutic components 34 and cellular components 36 act as a source of one or more therapeutic agents 40 when coated stent 10 is deployed in vessel 50 of mammalian body 52. Therapeutic agents 40 may elute from alginate coating 30 through endoluminal wall 56 of vessel 50 and into various tissues of stenosed region 54 and vessel 50 near the deployed stent.

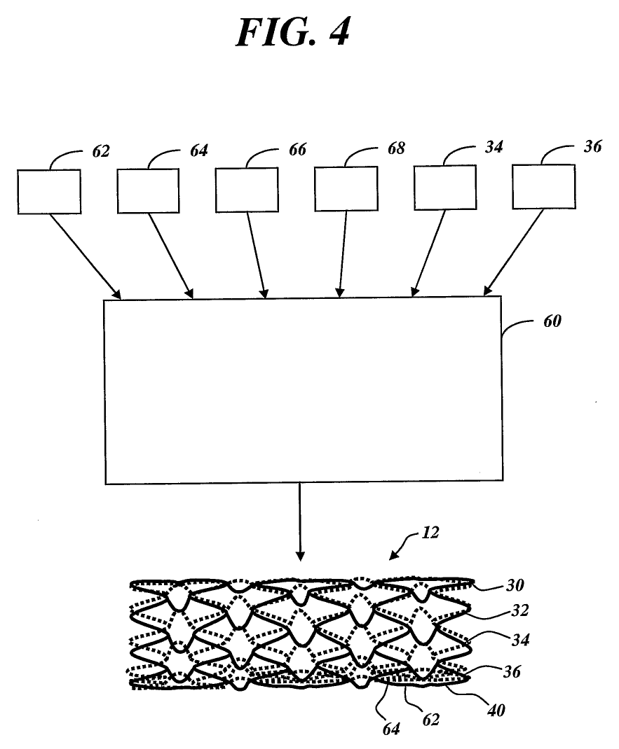

FIG. 4 is a schematic diagram of a method for coating an implantable medical device with a gel, in accordance with aspects of the present teachings. An alginate or other gel-based coating 30 for an implantable medical device 12 may include an alginate (or other gel) matrix 32 and a therapeutic component 34 dispersed within alginate matrix 32. Alternatively, or in addition, alginate coating 30 for implantable medical device 12 includes alginate matrix 32 and cellular component 36 dispersed within alginate matrix 32. Alginate coating 30 may contain one or more therapeutic components 34 and cellular components 36 dispersed within alginate matrix 32.

Alginate coating 30 is formed or otherwise deposited on exposed portions of implantable medical device 12 to provide, for example, mechanical protection and controlled, time-release delivery of therapeutic agents 40 from either therapeutic components 34 or cellular components 36 dispersed within alginate coating 30. In some embodiments, alginate coating 30 with alginate matrix 32 may encapsulate and maintain the viability of cellular components 36, allowing therapeutic agents 40 produced by the cells to pass through alginate matrix 32 and elute into surrounding target tissues such as arterial tissues.

A ratio of mannuronate alginate subunits 62 and guluronate alginate subunits 64 may be selected to provide a predetermined elution characteristic of the alginate coating.

An alginate premix of mannuronate alginate subunits 62 and guluronate alginate subunits 64 (in any suitable polymerized (or nonpolymerized) form), an alginate solvent 66 such as alcohol or water, and one or more therapeutic components 34 and cellular components 36 may be combined to form an alginate solution with the determined ratio of mannuronate alginate subunits 62 and guluronate alginate subunits 64, in a fluent form. The alginate subunits may be provided as polymer chains (generally not yet substantially cross-linked) or shorter oligomers (or individual subunits). The term “monomer” as used herein, is intended to mean a subunit moiety, whether the subunit moiety is part of a linear polymer chain, a three-dimensional network, or not linked to other subunits. An alginate linking agent 68 may be added to alginate solution 60, to cross-link the chains. Implantable medical device 12 such as a stent latticework may be coated with alginate solution 60, where the alginate gels to form a gel coating on external surfaces of implantable medical device 12.

Alginate coating 30 may be coated onto implantable medical device 12 (an implant) such as a stent, a valve, a pacemaker lead, a pacemaker, a pacing device, a venous filter, an abdominal aortic abdominal aneurysm device, or a vascular graft. Alternatively, a gel, such as alginate may form the implant body of an implant.

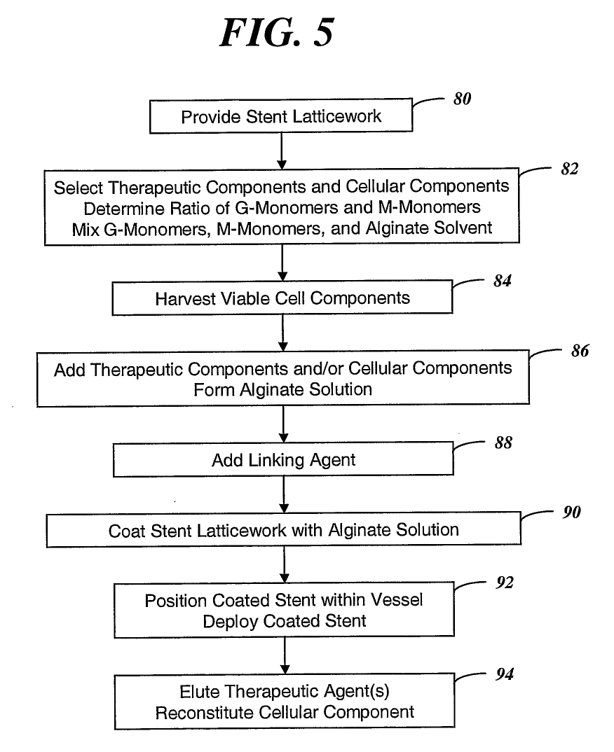

FIG. 5 is a flow diagram of a method of treating a vessel in a mammalian body, in accordance with one embodiment of the present invention. Treatable vessels include, for example, a coronary vessel, a cardiovascular vessel, a carotid artery, a hepatic vein, a hepatic artery, an artery, a vein, a peripheral vessel, an esophagus, a bile duct, a trachea, an intestine, a urethra, or a colon. The method includes various steps to form a coated stent or other implantable medical device and to treat or prevent a medical condition in the vessel. Fabrication of the coated stent may occur remotely to, or in some cases, within a clinical setting so that cells may be harvested from a donor or recipient and combined with the coating material immediately prior to implantation of the device in the recipient.

A stent latticework is provided, as seen at block 80. The stent latticework may be balloon-expandable or self-expandable, and may have a stent body including a metal such as stainless steel, nitinol, platinum, or a biocompatible metal alloy. Alternatively, the stent latticework may have a polymeric body comprised of a polymer such as poly-L-lactide. The length, expanded diameter, and compressed diameter of the stent may be selected in accordance with the vessel to be stented.

The desired therapeutic components and/or cellular components may be selected, as seen at block 82. Selectable therapeutic components and cellular components may include any combination of the components described elsewhere in the present teachings.

Based on the desired elution characteristics of therapeutic agents from the therapeutic and cellular components, the ratio of mannuronate alginate monomers and guluronate alginate monomers may be determined. For example, the block length of mannuronate alginate subunits and the block length of guluronate alginate subunits may be selected to achieve suitable strength and flexibility of the coated device, while providing controlled delivery of therapeutic agents from the therapeutic and cellular components dispersed within the alginate matrix. The dose and constituency of added therapeutic and cellular components may be selected based on the desired treatment of the vessel.

In some examples, an alginate premix may be sterilized by its passage through a selection of submicron filters, by exposure to radiation in the form of ionizing gamma or electron beams, or by other known methods of rendering a viscous solution sterile. The premix may be mixed in a solution prior to filtration and then dried, for example, by dialysis or spray drying.

In another example, the mannuronate alginate subunits, guluronate alginate subunits, and an alginate solvent such as alcohol or water may be mixed to form the alginate solution with the determined ratio of mannuronate alginate subunits and guluronate alginate subunits. The concentration and viscosity of the alginate solution may be reduced with the addition of aqueous cellular or therapeutic components.

In an optional step, one or more viable cell components may be harvested from a host or donor mammalian body, as seen at block 84. The harvested viable cellular component comprises, for example, endogenous endothelial cells. The harvested cells may be further cultured to increase their numbers or further filtered to obtain the desired quantity, quality, and type of cell. In some examples, the harvested viable cellular component may be mixed into the alginate solution prior to coating the stent latticework. In some examples, freeze-dried cells may be mixed into the alginate solution with, for example, an aqueous-based alginate solvent. The freeze-dried cells may be reconstituted when the coated stent is inserted and deployed in the mammalian body.

The selected therapeutic components and cellular components may be mixed with the determined ratio of mannuronate alginate subunits and guluronate alginate subunits or the alginate premix to form the alginate solution prior to coating the stent latticework, as seen at block 86. For example, endothelial cells may be mixed into a formulation of alginate with appropriate mannuronate and guluronate components into an alginate solution, and the stent is coated with the cellularized alginate solution.

In some examples, an alginate linking agent is added to the alginate solution, as seen at block 88. The added alginate linking agent comprises, for example, divalent calcium, divalent barium, divalent strontium, divalent magnesium, or a source of calcium such as a calcium salt. The alginate linking agent may be added to the alginate solution immediately prior to coating the stent latticework or other implantable medical device, due to rapid gelling and setting of the alginate matrix. The alginate matrix is cross-linked, for example, with a divalent-cation solution such as a calcium solution. In another example, the alginate linking agent is applied to the stent latticework prior to the application of the alginate solution, and as it is applied, the alginate solution coagulates onto the stent latticework. In another example, the alginate linking agent is applied to a stent latticework previously coated with the alginate solution, causing the alginate solution to gel and harden accordingly. In another example, alternating alginate layers with varying ratios of mannuronate and guluronate monomers are incorporated onto the stent latticework, with an optional capping coat that is abrasion and/or tear-resistant. An alginate linking agent in a solution may be applied, for example, by dipping the alginate-coated device in a bath of divalent cation solution or by spraying the divalent cation solution onto the coated stent to initiate cross-linking, gelling and hardening. An alginate coating with multiple layers may be formed from successive dips into the same or different alginate solutions. Cross-linking and polymerization of the alginate solution may be activated at room temperature, or with exposure to ultraviolet light, infrared light, or thermal energy.

The stent latticework is coated with an alginate solution to form a coated stent having an alginate coating disposed on the stent latticework, as seen at block 90. The alginate coating may include one or more therapeutic components or cellular components. The stent latticework may be coated by, for example, spraying, dipping, and rolling the stent latticework with the alginate solution at temperatures below, for example, 37 degrees centigrade. The alginate solution includes a plurality of alginate monomers and an alginate solvent, and may include one or more therapeutic components or cellular components. The coated stent is dried and loaded onto a suitable catheter delivery system. The resulting device can be sterilized with conventional means that do not alter or damage the therapeutic or cellular components or the alginate matrix.

When used in a medical procedure, the coated stent is positioned within a vessel and deployed, as seen at block 92. Positioning of the coated stent is accomplished, for example, by coupling the coated stent onto a delivery catheter, and advancing the coated stent to a treatment area by using a guidewire, as is known in the art. The coated stent is deployed (expanded), for example, by inflating and expanding an inflation balloon coupled to near the distal end of the catheter, or by retracting a sheath from a self-expanding stent latticework.

Once deployed, one or more therapeutic agents may be eluted from the alginate coating, as seen at block 94. The alginate coating controls aspects of the elution (such as rate, direction, etc.) of the therapeutic agent when the coated stent is deployed. In one example, the eluted therapeutic agent comprises nitric oxide from entrained endothelial cells to regulate the proliferation of smooth muscle cells in the vessel near the deployed stent. In another example, the cellular component in the alginate solution is reconstituted when the coated stent is deployed, and therapeutic agent is produced and delivered to the vessel.

IV. Formation of Implants In Situ

Implants (such as stents and/or caps, among others) for vessels or other lumens may be formed in situ within an implant recipient. The implants may, for example, provide support in a vessel (stents) and/or cap or cover plaque (caps).

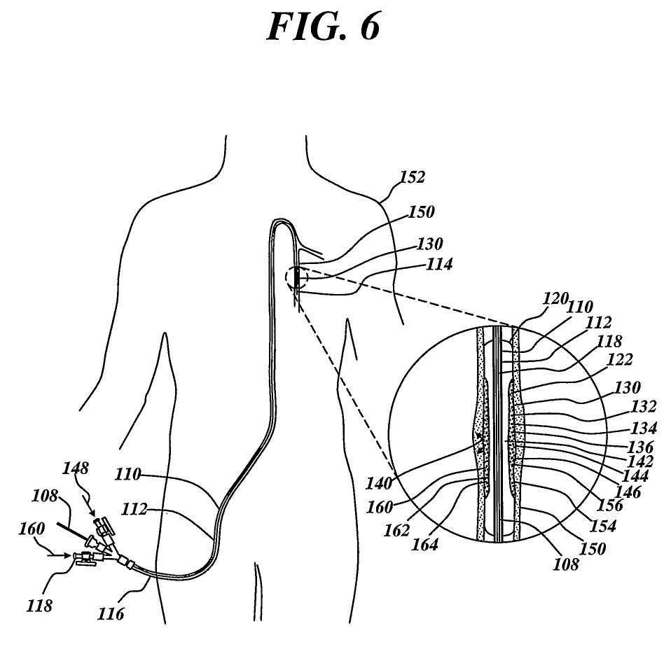

FIG. 6 illustrates a system for treating a vessel 150 in a mammalian body 152, in accordance with aspects of the present teachings. The system may include an implant formation catheter 110 having a catheter body 112. One or more inflatable balloons such as a formation balloon 120 may be attached to catheter body 112 near a distal end 114 of catheter body 112. An alginate stent 130 (and/or a cap) may be formed from an alginate solution 160 injected through an alginate-delivery lumen 118 included within catheter body 112 into a portion 156 of vessel 150. Alginate solution 160 is injected into a cavity 122 between formation balloon 120 and an endoluminal wall 154 of vessel 150 when formation balloon 120 is inflated. An alginate cap may be formed, for example, to treat vulnerable plaque and/or inflamed tissue adjacent a lumen. Stents and/or caps may be formed with or without openings in their walls and may have any suitable thickness. Accordingly, implants may provide a support function to keep a vessel (or other lumen) open and/or may release therapeutic agents to the vessel or other tissue.

The formed alginate stent (or other implant) 130 may include a gel, for example, an alginate matrix (or other gel matrix) 132 in contact with endoluminal wall 154 of vessel 150, and a central lumen 142 axially extending through alginate matrix 132.

Formation balloon 120 may have surface features 146 to form at least one aperture 144 in alginate stent 130 when alginate solution 160 is injected. Alginate stent 130 may have one or more apertures 144 formed in alginate matrix. Apertures 144 may be positioned between central lumen 142 of alginate stent 130 and endoluminal wall 154 of vessel 150. Alternatively, the implant may be formed without apertures, such as a lining or cap to cover vulnerable plaque.

Inflation lumens within the catheter body 112 allow an inflation fluid 148 to be transported from a proximal end 116 of stent formation catheter 110 into and out of the interior regions of one or more inflation balloons attached to catheter body 112. When stent formation catheter 110 is appropriately positioned within vessel 150, exemplary alginate stent 130 is formed by inflating formation balloon 120, creating a cavity 122 between an outer surface of formation balloon 120 and endoluminal wall 154 of vessel 150. A guidewire 108 may be used to position stent formation catheter 110 at a desired location in mammalian body 152, as is known in the art. To form a cap, the implant-formation catheter 10 may have an over-the-wire, rapid exchange, monorail, or other type of catheter configuration, as is known in the art. An alginate solution 160 is injected through a port at proximal end 116, through alginate-delivery lumen 118, and into cavity 122, where it hardens (gels such as by cross-linking) to form alginate stent 130 against endoluminal wall 154 of the vessel. Alginate stent 130 provides mechanical support for vessel 150, as well as elutes and locally delivers one or more therapeutic agents 140.

Alginate stent 130 can support and treat vessel 150 in mammalian body 152. Alginate stent 130 may be used, for example, in a coronary vessel, a cardiovascular vessel, a carotid artery, a hepatic vein, a hepatic artery, an artery, a vein, a peripheral vessel, an esophagus, a bile duct, a trachea, an intestine, a urethra, or a colon.

Alginate stent 130 provides a mechanism for controlled, time-release characteristics of therapeutic agents 140 from any therapeutic components 134 and cellular components 136 within an alginate matrix 132 of alginate stent 130. In one embodiment, the invention provides localized delivery of one or more therapeutic agents 140 from therapeutic components 134 dispersed within alginate stent 130 when alginate stent 130 is formed within a vessel 150 of the mammalian recipient. In another embodiment, the invention provides long-term delivery of one or more therapeutic agents 140 via an alginate matrix 132 suitable for maintaining encapsulated cells and aggregates of viable cells from transplanted or implanted cells that produce such therapeutic agents.

Alginate stent 130 may include one or more therapeutic components 134 and/or cells dispersed within alginate matrix 132. Any suitable therapeutic components and/or cells may be included. Exemplary therapeutic components and cells that may be suitable are described in Section II and elsewhere in the present teachings.

Alginate matrix 132 may include selected therapeutic components 134 and cellular components 136 that produce therapeutic agents 140 for elution from alginate matrix 132 of alginate stent 130. When cellular components 136 are selected, alginate matrix 132 serves as an immune barrier so that the immune system of the recipient does not recognize and destroy cellular component 136 contained within alginate matrix 132, or terminate the production of therapeutic agents 140. Meanwhile, alginate matrix 132 still allows for the metabolic transfer of nutrients, wastes, and therapeutic proteins and agents to pass through alginate matrix 132 into surrounding vessel 150. Therapeutic agents 140 are delivered in close proximity to the treatment site and released from alginate stent 130. Alginate stent 130 with therapeutic components 134 and cellular components 136 provides long-term expression of the therapeutic agents 140.

Alginate stent 130 having therapeutic components 134 or cellular components 136 may help prevent restenosis by eluting of one or more therapeutic agents 140 near the tissue needing treatment. For example, the eluted therapeutic agents may regulate proliferation of smooth muscle cells in the vicinity of alginate stent 130, or inhibit fibrin formation and growth of neointimal tissue within the treated area of vessel 150.

Living cells or other biomaterials and therapeutic compounds may be immobilized in alginate matrix 132 such as an alginate gel. Cells immobilized in alginate gels maintain good viability during long-term culture, due in part to the mild environment of the gel network. Alginate gel provides a physically protective barrier for immobilized cells and tissue, and inhibits immunological reactions of the host. Alginate matrix 132 provides a location that is viable and productive for cellular components 136, since alginate matrix 132 allows the diffusion of nutrients to the cell, diffusion of respiratory byproducts to the surrounding area, and diffusion of selected therapeutic components 134 in an unaltered condition from alginate matrix 132. In some cases, alginate matrix 132 serves as an immune barrier while providing for diffusive transport for therapeutic and cellular materials. The immune barrier properties of alginate matrix 132 are particularly useful for non-host derived cell sources, or manipulated cells of designer deoxyribonucleic acid (DNA).

One example of a cellular component 136 is an endothelial cell that produces nitric oxide, a regulating molecule for smooth muscle cell quiescence and maintenance of vascular smooth muscle cells in the non-proliferative stage. A patient's own endothelial cells from, for example, microvascular adipose tissue, may be harvested and mixed with an alginate solution, and formed along with alginate matrix 132 into alginate stent 130. Upon implantation, the endothelial cells remain viable and locally produce nitric oxide to regulate and maintain the quiescent nature of smooth muscle cells, which can be a contributor to the production and recruitment of fibroblasts from the media and adventitia of arteries. With the continued long-term production of nitric oxide from the translocated endothelial cells, vascular patency may be maintained for a period substantially longer than the period for potential stenotic reoccurrence following stent formation.

Long-term administration of at least one therapeutic agent 140 such as nitric oxide may be provided to vessel 150. Disruption of the endothelial lining in vessel 150 may result in the reduction of nitric oxide production, leading to the loss of regulation of the smooth muscle cells. This disruption can occur during placement of conventional stents, angioplasty procedures, or from disease accumulation. Stent placement and angioplasty procedures that open an occluded vessel exert significant pressure on the luminal surface and may damage the endothelial cells.

Since it is such a small molecule, nitric oxide is able to diffuse rapidly across cell membranes and, depending on the conditions, is able to diffuse distances of more than several hundred microns, as is demonstrated by its regulation of smooth muscle cells, vascular dilation, tissue compliance and physiological tone of the vessel. Nitric oxide may be produced within alginate matrix 132 and delivered directly to the vessel. For example, L-arginine, a naturally occurring amino acid, and other nutraceuticals may be converted to nitric oxide within alginate matrix 132 by a group of enzymes such as nitric oxide synthases. These enzymes convert L-arginine into citrulline, producing nitric oxide in the process. In another example, nitric oxide is liberated from diazeniumdiolates, compounds that release nitric oxide into the blood stream and vascular walls.

Alginate stent 130 comprises alginate matrix 132 with, for example, cross-linked chains of mannuronate alginate monomers 162 and guluronate alginate monomers 164. A predetermined ratio of mannuronate alginate monomers 162 and guluronate alginate monomers 164 can be selected and formed into alginate matrix 132 to provide the desired elution rates for therapeutic agents 140.

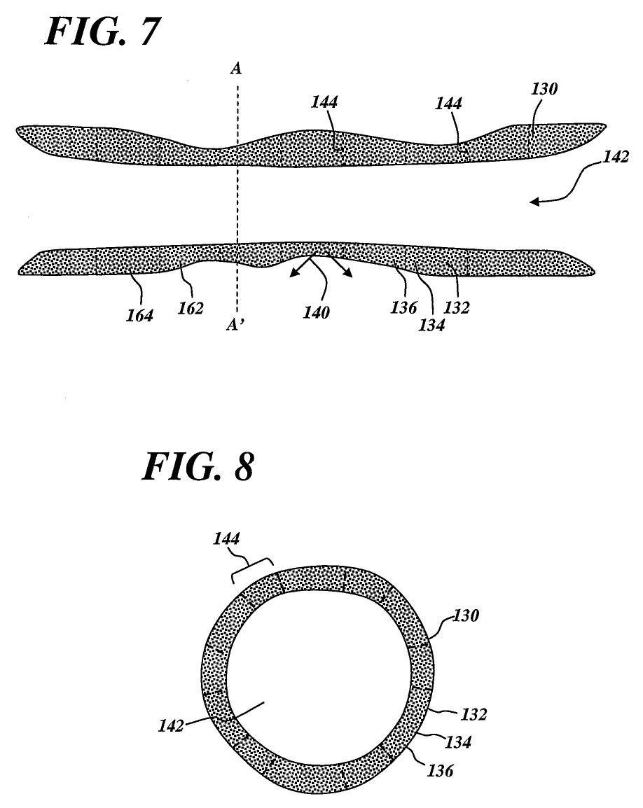

FIG. 7 illustrates a longitudinal view of an exemplary alginate stent, in accordance with one embodiment of the present invention. FIG. 8 illustrates an axial sectional view of the alginate stent of FIG. 7, with like-numbered elements referring to similar or identical elements in each illustration. FIG. 7 and FIG. 8 taken together, an alginate stent 130 includes an alginate matrix 132 and a central lumen 142 axially extending through alginate matrix 132. Alginate stent 130 may include one or more therapeutic components 134 and/or cellular components 136. Therapeutic components 134 and cellular components 136 may be dispersed uniformly within alginate matrix 132 or have a preferred distribution. Therapeutic agents 140 are eluted from alginate stent 130, wherein alginate matrix 132 controls the elution of therapeutic agents 140. Alginate stent 130 provides a mechanism for controlled, time-release characteristics of therapeutic agents 140 from any therapeutic components 134 and cellular components 36 within an alginate matrix 132 of alginate stent 130. In one embodiment, the invention provides localized delivery of one or more therapeutic agents 140 from therapeutic components 134 dispersed within alginate stent 130 when alginate stent 130 is deployed within a vessel of a mammalian recipient. In another embodiment, the invention provides long-term delivery of one or more therapeutic agents 140 via a matrix suitable for maintaining encapsulated cells and aggregates of viable cells from transplanted or implanted cells that produce such therapeutic agents.

An array of apertures 144 may be included in alginate stent 130 to provide support for the vessel wall while allowing transport of material through the sides of alginate stent 130.

Alginate stent 130 may have cross-linked chains of mannuronate alginate monomers 162 and guluronate alginate monomers 164 in a predetermined ratio to provide the desired mechanical strength and flexibility while controlling the elution rates for therapeutic agents 140 from alginate stent 130.

FIG. 8 illustrates an axial cross-sectional view of the alginate stent of FIG. 7, taken through line A-A′. Alginate stent 130 includes an alginate matrix 132 that may have one or more therapeutic components 134 or cellular components 136 dispersed therein. For example, therapeutic components 134 and cellular components 136 dispersed within alginate stent 130 may be uniformly dispersed throughout, have a non-uniform profile with a higher concentration of therapeutic components 134 or cellular components 136 nearer the central lumen 142, or have a non-uniform profile with a higher concentration of therapeutic components 134 and cellular components 136 closer to an outer surface of alginate stent 130. In another example, therapeutic components 134 and cellular components agglomerate or collect in regions within alginate stent 130. One or more apertures 144 may be included in alginate stent 130 to provide support for the vessel wall while allowing transport of material through the sides of alginate stent 130.

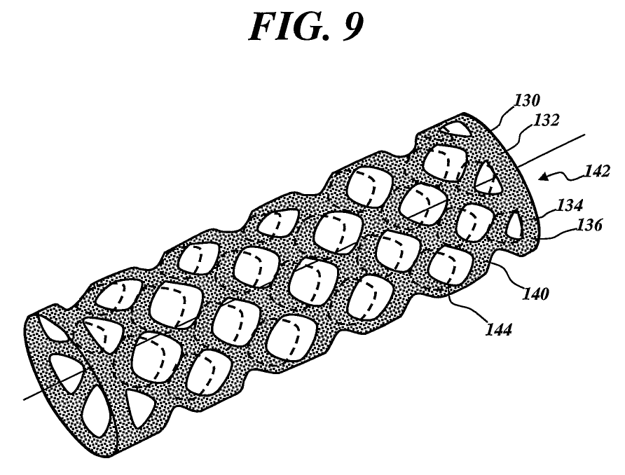

FIG. 9 illustrates an alginate stent 130 with a central lumen 142 and a plurality of apertures 144, in accordance with one embodiment of the present invention. Alginate stent 130 may have one or more apertures 144 formed in an alginate matrix 132 of alginate stent 130, to allow, for example, the transport of nutrients to and waste materials from vessel or organ walls. An aperture 144 may be included in alginate stent 130 to allow blood or other bodily fluid to flow through, for example, a vessel that is bifurcated with a branching vessel, which would otherwise be blocked by the formation of a more solid tubular form of alginate stent 130. An array of apertures 144 may be included in alginate stent 130 to provide support for the vessel wall while allowing transport of material through the sides of alginate stent 130. Alternatively, the implant may be a cap for vulnerable plaque, which may include or lack apertures.

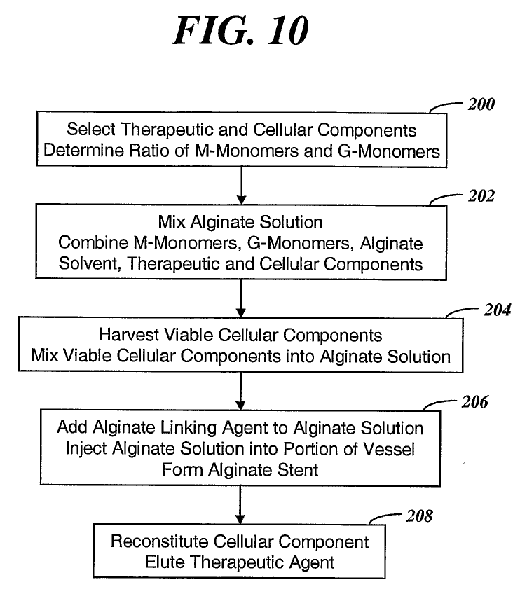

FIG. 10 is a flow diagram of a method for treating a vessel in a mammalian body, in accordance with another embodiment of the present invention. The method includes various steps to form an alginate stent and to treat or prevent one or more medical conditions in the region of alginate stent formation. The alginate stent includes an alginate matrix, and one or more therapeutic components and cellular components may be dispersed therein. Treatable vessels include, for example, a coronary vessel, a cardiovascular vessel, a carotid artery, a hepatic vein, a hepatic artery, an artery, a vein, a peripheral vessel, an esophagus, a bile duct, a trachea, an intestine, a urethra, or a colon. Formation of the alginate stent may occur in a clinical setting, so that donor-provided cells, for example, may be harvested from a host or donor mammalian body and combined into the alginate solution immediately prior to formation of the alginate stent. The harvested cells may be further cultured to increase their numbers or further filtered to obtain the desired quantity, quality and type of cells.

The alginate stent is formed within a vessel to provide mechanical support and controlled, time-released delivery of therapeutic agents from either therapeutic components or cellular components dispersed within the alginate stent. In one embodiment, the alginate stent with an alginate matrix encapsulates and maintains the viability of cellular components, and allows the expression of therapeutic agents from the cells to pass through the alginate matrix and elute into surrounding target tissues such as arterial tissues. The alginate matrix and therapeutic or cellular components may be used in conjunction with various medical procedures using vascular devices such as abdominal aortic aneurysm (AAA) devices, venous filters, vascular grafts, and valves.

Desired therapeutic components and cellular components are selected along with the desired quantity, as seen at block 200. Selectable therapeutic components and/or cellular components may include any of the source components and/or therapeutic agents described in Section II or elsewhere in the present teachings. Selectable cellular components include, for example, endothelial cells, designer-DNA manipulated cells, host-derived cells from a host source, donor-derived cells from a donor source, pharmacologically viable cells, freeze-dried cells, or a combination thereof. The dose and constituency of added therapeutic and cellular components may be selected based on the desired treatment of the vessel and the desired elution rate of the therapeutic agents.

A ratio of mannuronate alginate monomers and guluronate alginate monomers may be determined to provide a predetermined elution characteristic of the alginate stent. Based on the desired elution characteristics of the therapeutic and cellular components, the ratio of mannuronate alginate monomers and guluronate alginate monomers may be determined. For example, the block length of mannuronate alginate monomers and the block length of guluronate alginate monomers are selected to achieve suitable strength and flexibility of the stent, while providing controlled delivery of therapeutic and cellular components dispersed within the alginate matrix.

Prior to injection and formation of the alginate stent, the alginate premix, monomers or polymers may be sterilized by passage through a selection of submicron filters, by exposure to radiation in the form of ionizing gamma or electron beams, or by other known methods of rendering a viscous solution sterile. The premix may be mixed in a suitable solvent prior to filtration and then dried, for example, by dialysis or spray drying.

An alginate solution including an alginate premix and an alginate solvent is mixed prior to forming the alginate stent, as seen at block 202. In one example, the mannuronate alginate monomers, guluronate alginate monomers, and an alginate solvent such as alcohol or water are mixed to form the alginate solution with the determined ratio of mannuronate alginate monomers and guluronate alginate monomers. The concentration and viscosity of the alginate solution may be reduced with the addition of aqueous cellular or therapeutic components. In another example, the mannuronate alginate monomers, guluronate alginate monomers, alginate solvent, and the selected therapeutic or cellular components are combined to form the alginate solution with the determined ratio of mannuronate alginate monomers and guluronate alginate monomers. For example, endothelial cells are mixed into a formulation of alginate with appropriate mannuronate and guluronate components into an alginate solution, and the alginate solution used to form the alginate stent. In another example, an alginate premix of mannuronate alginate monomers and guluronate alginate monomers, an alginate solvent such as alcohol or water, and one or more therapeutic components and cellular components are combined to form the alginate solution.

A radiopaque additive such as divalent barium may be added to the alginate solution to improve fluoroscopic and radioscopic visualization of the alginate solution during formation of the alginate stent within the mammalian body. In some examples, the radiopaque additive may be a cross-linking agent for stimulating gel-formation.

In an optional step, one or more viable cell components may be harvested from the host or a donor mammalian body, and incorporated or otherwise mixed into the alginate solution prior to formation of the alginate stent in the mammalian body, as seen at block 204. The harvested cells may be further cultured to increase their numbers or further filtered to obtain the desired quantity, quality and type of cells. The harvested viable cellular component, such as endogenous endothelial cells, is mixed into the alginate solution prior to injecting the alginate solution. In another example, freeze-dried cells are mixed into the alginate solution with for, example, an aqueous-based alginate solvent. The freeze-dried cells are reconstituted when the alginate stent is formed within the mammalian body. In another example, cells from either a host or donor source are preserved with trehalose and freeze-dried, rendering the cells functional yet in a dehydrated state. Use of cells in a preserved fashion allows for mixing the alginate solution with the cells in advance or conjointly with the medical procedure. One skilled in the art can identify alternative cell-producing components that can be substituted for endothelial cells and provide therapeutic products from the alginate matrix.

An alginate linking agent is added to the alginate solution, as seen at block 206. The added alginate linking agent comprises, for example, divalent calcium, divalent barium, divalent strontium, divalent magnesium, or a source of calcium such as a calcium salt. In one example, the alginate linking agent is added to the alginate solution immediately prior to injecting the alginate solution, due to rapid gelling and setting of the alginate matrix. In another example, the alginate linking agent is added to the alginate solution after injecting the alginate solution into the portion of the vessel. In another example, the alginate linking agent is co-injected into a portion of the vessel to form the stent. In another example, the alginate linking agent is injected into the stent-formation cavity and combined with alginate solution injected from a separate port. In another example, the alginate linking agent is deposited, applied, diffused, or otherwise transferred to an endoluminal wall of the vessel prior to injecting the alginate solution into the portion of the vessel. As the alginate solution is injected, the alginate solution coagulates onto the vessel wall. Cross-linking and polymerization of the alginate solution may occur in situ while at mammalian body temperature, or activated with exposure to ultraviolet light, infrared light, or thermal energy.

The alginate solution is injected into a cavity formed within a portion of the vessel, where the alginate solution cross-links, gels, and hardens to form the alginate stent. The alginate stent is formed in contact with an endoluminal wall of the vessel and has a central lumen axially extending through the alginate stent. The amount of alginate solution injected into the cavity is related to the length and thickness of the formed stent.

The alginate solution may be injected into a portion of the vessel with a stent formation catheter. The stent formation catheter is positioned, for example, by advancing the distal end of the stent formation catheter to a treatment site using a guidewire inserted into the vessel, as is known in the art When the stent formation catheter is positioned, the alginate stent may be formed with one or more formation balloons attached to the catheter body. The formation balloon may have surface features to form one or more apertures in the alginate stent when the alginate solution is injected.

Once the alginate stent is formed, one or more therapeutic agents may be eluted from therapeutic or cellular components dispersed within the alginate stent, as seen at block 208. In one example, the eluted therapeutic agent comprises nitric oxide from entrained endothelial cells to regulate the proliferation of smooth muscle cells in the vessel near the formed alginate stent. In another example, the cellular component in the alginate solution is reconstituted after the cellularized alginate stent is formed in the vessel, and therapeutic agents are produced and delivered to the vessel from the reconstituted cellular component. The immune barrier of the alginate matrix protects the cellular components. The alginate matrix of the alginate stent controls the elution of the therapeutic agent from therapeutic and cellular components within the matrix.

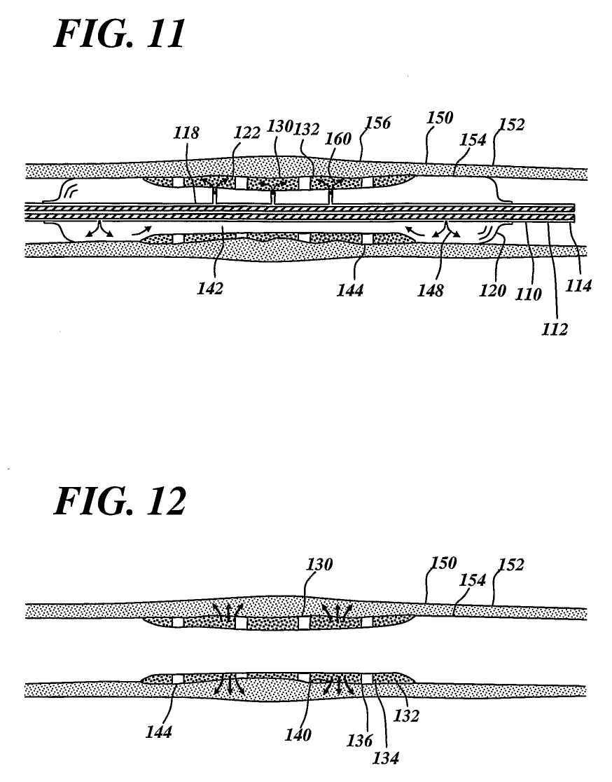

FIG. 11 illustrates a longitudinal sectional view of an alginate stent 130 being formed within a vessel 150 of a mammalian body 152, in accordance with one embodiment of the present invention. Vessel 150 has a partial occlusion or stenosed portion 156 that blocks the flow of fluid through vessel 150. A stent formation catheter 110 with a catheter body 112 has a dog-boned formation balloon 120 attached to catheter body 112 near a distal end 114 of catheter body 112. Dog-boned (dumbbell-shaped), as used herein, means widened at opposing end regions relative to a central region disposed between the end regions. Formation balloon 120 is inflated, for example, with contrast fluid or inflation fluid 148 injected into an interior region of formation balloon 120. An alginate-delivery lumen 118 within catheter body 112 delivers an alginate solution 160 into a cavity 122 formed between formation balloon 120 and an endoluminal wall 154 of vessel 150 when formation balloon 120 is inflated. Formation balloon 120 may have surface features to form one or more apertures 144 in alginate stent 130 when alginate solution 160 is injected. Slots, grooves or flexible tubes are used, for example, to guide alginate solution 160 from alginate-delivery lumen 118 into cavity 122.

As alginate solution 160 sets and hardens, alginate stent 130 with alginate matrix 132 and a central lumen 142 is formed within vessel 150 of mammalian body 152. With alginate stent 130 formed in the stenosed region, endoluminal walls 154 may be locally expanded outward to reduce the constriction and allow for increased fluid flow through the vessel.

FIG. 12 illustrates a longitudinal cross-sectional view of an alginate stent 130 formed within a vessel 150 of a mammalian body 152, in accordance with one embodiment of the present invention. Alginate stent 130 includes an alginate matrix 132 in contact with an endoluminal wall 154 of vessel 150. Therapeutic agents 140 may be eluted from alginate stent 130 from one or more therapeutic components 134 and cellular components 136 dispersed within alginate matrix 132. Eluted therapeutic agents 140 migrate into endoluminal wall 154 and other tissues near alginate stent 130 to provide desired therapeutic effects. Alginate stent 130 may have one or more apertures 144 formed in alginate matrix 132 of alginate stent 130.

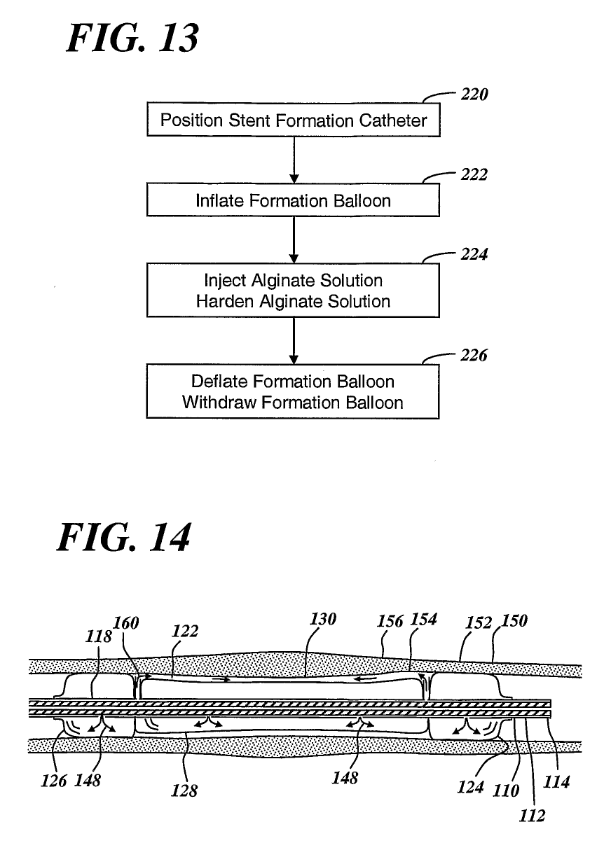

FIG. 13 is a flow diagram of a method of forming an alginate stent in a vessel of a mammalian body, in accordance with one embodiment of the present invention. The method includes various steps to form an alginate stent 130 as described with respect to FIG. 11 and FIG. 12.

Stent formation catheter 110 is positioned within vessel 150, as seen at block 220. Stent formation catheter 110 has catheter body 112 with alginate-delivery lumen 118. Exemplary catheter body 112 has an inflation lumen for transporting inflation fluid 148 to inflate formation balloon 120, and a guidewire lumen to aid in positioning stent formation catheter 110 within the mammalian body.

Formation balloon 120 attached to catheter body 112 near a distal end 114 of catheter body 112 is inflated, as seen at block 222. An inflation fluid or contrast fluid may be injected into formation balloon 120 to inflate and enlarge formation balloon 120.

An alginate solution 160 is injected through alginate-delivery lumen 118 into cavity 122 formed between inflated formation balloon 120 and endoluminal wall 154 of vessel 150, as seen at block 224. Alginate solution 160 is hardened with an alginate linking agent to form alginate stent 130 within vessel 150.

After alginate stent 130 has been formed, formation balloon 120 is deflated and withdrawn from vessel 150 along with stent formation catheter 110, as seen at block 226.

FIG. 14 illustrates a longitudinal sectional view of an alginate stent 130 being formed within a vessel 150 of a mammalian body 152, in accordance with another embodiment of the present invention.

Alginate stent 130 is formed in a vessel 150 of mammalian body 152 with a system that includes a stent formation catheter 110 having a catheter body 112. A distal occlusion balloon 124 is attached to catheter body 112 near a distal end 114 of catheter body 112. A proximal occlusion balloon 126 is attached to catheter body 112 proximal to distal occlusion balloon 124. A medial formation balloon 128 is attached to catheter body 112 between distal occlusion balloon 124 and proximal occlusion balloon 126. An alginate-delivery lumen 118 contained within catheter body 112 carries alginate solution 160 to treatable portion 156 of vessel 150. Alginate stent 130 is formed from an alginate solution 160 injected through alginate-delivery lumen 118 into a cavity 122 between medial formation balloon 128 and an endoluminal wall 154 of vessel 150 when distal occlusion balloon 124 and proximal occlusion balloon 126 are inflated with an inflation fluid 148. Slots, grooves or flexible tubes may be used to guide alginate solution 160 from alginate-delivery lumen 118 into cavity 122. Medial formation balloon 128 may have surface features (not shown) to form one or more apertures in alginate stent 130 when alginate solution 160 is injected.

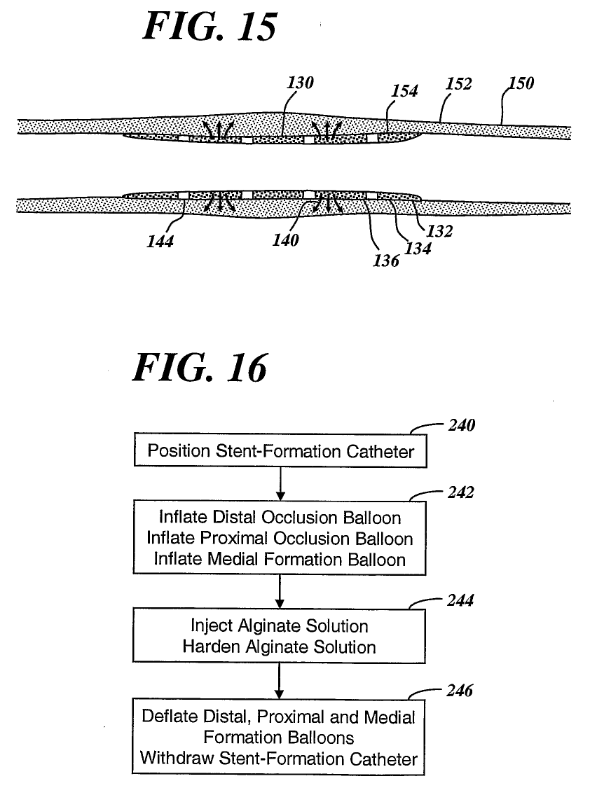

FIG. 15 illustrates a longitudinal sectional view of an alginate stent 130 formed within a vessel 150 of a mammalian body 152, in accordance with another embodiment of the present invention. Alginate stent 130 includes an alginate matrix 132 in contact with an endoluminal wall 154 of vessel 150, and may include one or more therapeutic components 134 or cellular components 136. Therapeutic agents 140 are eluted from therapeutic components 134 and cellular components 136 dispersed within alginate matrix 132 of alginate stent 130. Therapeutic agents 140 elute from alginate stent 130 (inward) into the vessel lumen and/or (outward) through endoluminal wall 154 of vessel 150 and into various tissues of vessel 150 near formed alginate stent 130. Alginate stent 130 may have one or more apertures 144 formed in an alginate matrix 132 of alginate stent 130.

FIG. 16 is a flow diagram of various steps of a method of forming alginate stent 130 in vessel 150 of mammalian body 152, in accordance with another embodiment of the present invention, and as described with respect to FIG. 14 and FIG. 15. Stent formation catheter 110 is positioned in vessel 150, as seen at block 240. Stent formation catheter 110 has catheter body 112, alginate-delivery lumen 118, and a plurality of inflation lumens.

Distal occlusion balloon 124 attached to catheter body 112 near distal end 114 of catheter body 112 is inflated, as seen at block 242. Proximal occlusion balloon 126, which is attached to catheter body 112 proximal to distal occlusion balloon 124, is inflated. Medial formation balloon 128 attached to catheter body 112 between distal occlusion balloon 124 and proximal occlusion balloon 126 is inflated. Distal occlusion balloon 124 and proximal occlusion balloon 126 are inflated to occlude vessel 150. Medial formation balloon 128 inflates to a diameter corresponding to the desired lumen diameter of alginate stent 130.

Alginate solution 160 is injected through alginate-delivery lumen 118 into cavity 122 formed between inflated distal occlusion balloon 124, inflated proximal occlusion balloon 126, inflated medial formation balloon 128, and endoluminal wall 154 of vessel 150, as seen at block 244. Alginate solution 160 hardens with an alginate linking agent to form alginate stent 130 within vessel 150.

When alginate stent 130 forms, distal occlusion balloon 124, proximal occlusion balloon 126, and medial formation balloon 128 are deflated, and stent formation catheter 110 is withdrawn from vessel 150, as seen at block 246.

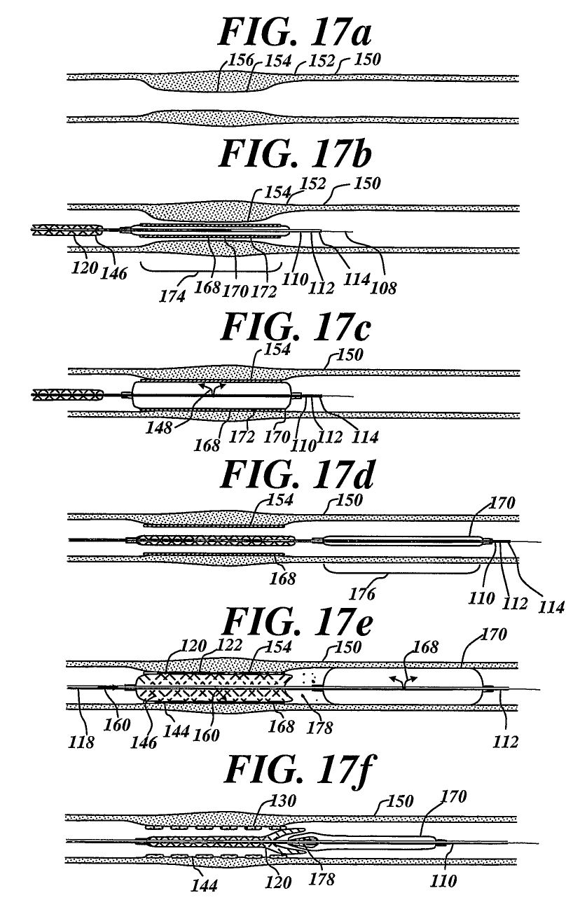

FIGS. 17a-f illustrate longitudinal sectional views of an alginate stent corresponding to steps of a method for forming an alginate stent 130, in accordance with another embodiment of the present invention. The illustrative steps are performed with an alginate stent formation system to treat a stenosed portion 156 a vessel 150 in a mammalian body 152. The system includes a stent formation catheter 110 having a catheter body 112. An angioplasty balloon 170 is attached to catheter body 112 near a distal end 114 of catheter body 112. Angioplasty balloon 170 has an alginate linking agent 168 disposed on a surface 172 of angioplasty balloon 170. A formation balloon 120 is attached to catheter body 112 proximal to angioplasty balloon 170. An alginate-delivery lumen 118 is included within catheter body 112. An alginate stent 130 is formed from an alginate solution 160 injected through alginate-delivery lumen 118 into a cavity 122 between formation balloon 120 and an endoluminal wall 154 of vessel 150 when formation balloon 120 is inflated. Formation balloon 120 may have surface features 146 to form at least one aperture 144 in alginate stent 130 when alginate solution 160 is injected.

Vessel 150 in mammalian body 152 having endoluminal wall 154 and one or more stenoses that locally block or restrict the flow of bodily fluid is illustrated in FIG. 17a. Stent formation catheter 110 is positioned at a first location 174 in vessel 150, as seen in FIG. 17b. Stent formation catheter 110 has a catheter body 112. A guidewire 108 inserted into mammalian body 152 may be used to guide stent formation catheter 110 to the desired position in vessel 150, as is known in the art.

Angioplasty balloon 170 attached to catheter body 112 near distal end 114 of catheter body 112 is inflated with an inflation fluid 148, as seen in FIG. 17c. When in contact with endoluminal wall 154, alginate linking agent 168 disposed on surface 172 of angioplasty balloon 170 is deposited on or otherwise transferred to endolurninal wall 154 of vessel 150. In an alternative embodiment, alginate linking agent 168 is pre-deposited on an outer surface of formation balloon 120, and transferred onto endoluminal wall 154 when formation balloon 120 is inflated.

Angioplasty balloon 170 is deflated, and stent formation catheter 110 is repositioned at a second location 176 in vessel 150, as seen in FIG. 17d. Second location 176, in this example, is distal to first location 174.

Angioplasty balloon 170 is re-inflated, as seen in FIG. 17e. Re-inflated angioplasty balloon 170 serves as a distal protection device. Formation balloon 120 attached to catheter body 112 proximal to angioplasty balloon 170 is inflated. Alginate solution 160 is injected through alginate-delivery lumen 118 into a cavity 122 formed between formation balloon 120 and endoluminal wall 154 of vessel 150. Slots, grooves or flexible tubes are used, for example, to guide alginate solution 160 from alginate-delivery lumen 118 into cavity 122. Alginate solution 160 flows around or through any surface features 146 to form apertures 144. Alginate solution 160 is hardened, for example, by alginate linking agent 168 deposited on endoluminal wall 154 of vessel 150.

Angioplasty balloon 170 and formation balloon 120 are deflated and withdrawn from vessel 150, as seen in FIG. 17f. Angioplasty balloon 170 may be configured to capture any embolic particles 178 when angioplasty balloon 170 and formation balloon 120 are deflated.

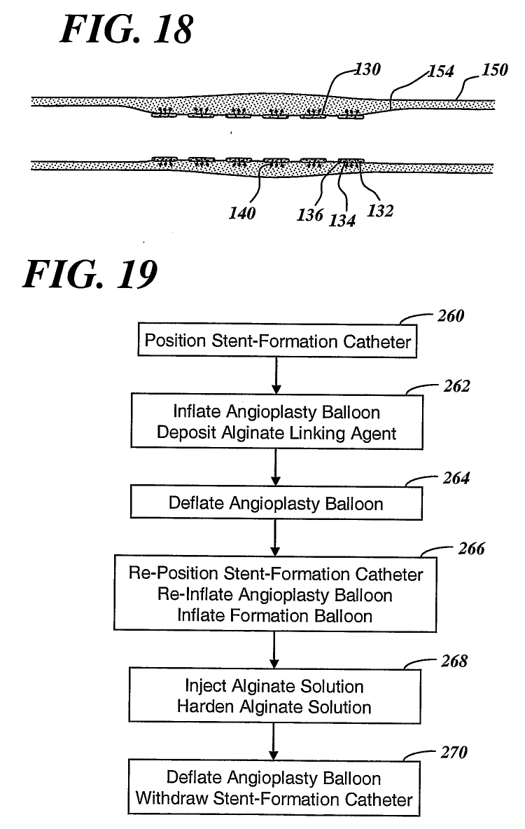

FIG. 18 illustrates a longitudinal sectional view of an alginate stent 130 formed within a vessel 150, in accordance with another embodiment of the present invention. Alginate stent 130 includes an alginate matrix 132 in contact with an endoluminal wall 154 of vessel 150. Therapeutic agents 140 are eluted from alginate stent 130 when one or more therapeutic components 134 and cellular components 136 are included within alginate matrix 132. Eluted therapeutic agents 140 migrate into endoluminal wall 154 and other tissues near alginate stent 130 to provide a therapeutic effect.

FIG. 19 is a flow diagram of steps in a method of, forming alginate stent 130 in vessel 150 of mammalian body 152, in accordance with another embodiment of the present invention and described with respect to FIG. 17 and FIG. 18.

Stent formation catheter 110 is positioned at first location 174 in vessel 150, as seen at block 260. Stent formation catheter 110 includes catheter body 112 with alginate-delivery lumen 118.

Angioplasty balloon 170 attached to catheter body 112 near distal end 114 of catheter body 112 is inflated with inflation fluid 148, as seen at block 262. Angioplasty balloon 170 has alginate linking agent 168 disposed on surface 172 of angioplasty balloon 170. Alginate linking agent 168 is deposited or otherwise transferred onto endoluminal wall 154 of vessel 150.

Angioplasty balloon 170 is deflated by withdrawing inflation fluid 148 from an interior region, as seen at block 264.

With angioplasty balloon 170 deflated to a reduced diameter, stent formation catheter 110 is repositioned at second location 176 located distally with respect to first location 174 in vessel 150, as seen at block 266. Angioplasty balloon 170 is re-inflated. Re-inflated angioplasty balloon 170 may serve as, for example, a distal protection device. A formation balloon 120 attached to catheter body 112 proximal to angioplasty balloon 170 is then inflated.

Alginate solution 160 is injected through alginate-delivery lumen 118 into cavity 122 formed between formation balloon 120 and endoluminal wall 154 of vessel 150, as seen at block 268. Alginate solution 160 is hardened or otherwise set to form alginate stent 130. Alginate linking agent 168 previously deposited onto endoluminal wall 154 of vessel 150 hardens alginate solution 160.

When alginate stent 130 is formed and hardened, angioplasty balloon 170 and formation balloon 120 are deflated and withdrawn from vessel 150, as seen at block 270. In one embodiment, angioplasty balloon 170 captures embolic particles 178 in a region of vessel 150 between angioplasty balloon 170 and formation balloon 120 when angioplasty balloon 170 and formation balloon 120 are deflated. For example, a proximal end of angioplasty balloon 170 encloses embolic particles 178 when deflated, and a distal end of formation balloon 120 encompasses the proximal end of angioplasty balloon 170 to retain embolic particles 178 while stent formation catheter 110 is being withdrawn. In another example, the proximal end of angioplasty balloon 170 includes a non-mobile calcium-rich surface that coagulates or cross-links any alginate residuals, effectively capturing the residuals. Alternatively, embolic particles 178 may be aspirated out of vessel 150, as is known in the art.

V. Formation of Bioreactors In Situ

This section describes formation of bioreactors in situ in a mammalian body.

A. Introduction

Various systems and therapeutic agents continue to be developed for improved long-term delivery of pharmacological and cellular therapeutics. Pills and injections are often ineffective means of administration for long-term treatments because constant drug delivery and higher local concentration are difficult to achieve via these means. Through repeated doses, drugs often cycle through concentration peaks and valleys, resulting in time periods of toxicity and ineffectiveness. In addition, dosages may be dispersed through the human body rather than being directed to a specific area where the treatment is needed.

Local and longer-term delivery of pharmacological and cellular agents at therapeutically effective levels is desirable for a number of medical procedures including those when medical devices are placed permanently within a human body. Drug-eluting coatings or sheaths for vascular stents, for example, are being developed to provide focused, local drug delivery. To increase the effectiveness of inhibitory drugs that are used for angioplasty and stent procedures, a relatively large number of drug molecules may need to be delivered into the intercellular spaces between smooth muscle cells of a vessel so that a therapeutically effective dose of molecules can cross cell membranes. The drug dosage may be difficult to control and direct into the proper intracellular compartments for treatment while minimizing intercellular redistribution of the drug throughout the body via the vascular system.

Long-term in-vivo cellular therapies are also being proposed as an alternative to traditional drug-delivery methods that use oral, intravascular or intramuscular introduction. For medical conditions where a person is unable to produce certain cells or the cells have been damaged, cellular therapeutics may provide long-term therapy. Cellular therapeutics employ living cells that deliver ameliorating natural or engineered biochemicals, or serve as full-scale replacements for defective tissues.

An early example and still widely used complex cellular therapeutics is human bone marrow transplantation as part of a defined treatment regime against leukemia. Since the late 1960s, bone marrow cells have been used to replace the chemotherapy-destroyed marrow of patients afflicted with cancer. These marrow cells can be derived either autologously from the patient before chemotherapy, or from other tissue donors. In some cases, cell therapies involve xenotransplantation of biological implants from completely different species.

A result of non-autologous transplantation is often the lifetime use of immunosuppressive drugs, unless the immune system can be retrained or diverted into accepting the new cells. For example, with pancreatic islet cell transplants, marrow cells from the donor of the islet cells are also transplanted into the host, thereby signaling the host immune system to modify itself and to accept the islet cells.

One proposed approach for eliminating the risk of cells being rejected by the host or the need to use anti-rejection drugs is to encapsulate cells in biocompatible polymeric substances. Intense study in animal models and human clinical trials have recently focused on encapsulating living cells for complex therapeutics, with clinical potential for the treatment of a wide range of diseases.

Cell microencapsulation is a technology where a living cell is infused or implanted in a microcapsule, which protects the cell from the immune system. A microcapsule needs sufficient permeability so that nutrients and oxygen can reach the transplanted cells, and appropriate cellular products, such as insulin from islet cells, can be released into the bloodstream or to adjacent tissues. At the same time, the capsular material should be restrictive enough to exclude immune cells and antibodies that can cause rejection and destroy the implant.

Various types of natural and synthetic polymers, particularly those having a semi-permeable aqueous characteristic, are being explored as encapsulation material. The success of an encapsulation material depends, at least in part, on its stability, chemical definability, lack of toxicity, permeability to oxygen and nutrients as well as the released therapeutic compounds, and its resistance to antibodies or cellular attack.

Materials for potential polymeric encapsulation systems include polysaccharide hydrogels, chitosan, calcium alginate or barium alginate. Photopolymerizable poly(ethylene glycol) (PEG) polymer and polyacrylates such as hydroxyethyl methacrylate methyl methacrylate, also have been proposed encapsulant materials. One encapsulation system employs photolithography techniques to encapsulate living cells in silicon nanocapsules, which have pores of a few nanometers.

A primary purpose for recent research on biocompatible semi-permeable membranes is to create a protective structure around therapeutic cells that grow in vivo and act as a miniature artificial organ or cell factory within the host body. The survival of encapsulated cells requires direct vascularization of the cells along with necessary nutrition and effective protection of the cells from the immune system. In some clinical applications, it is important for a cellular factory to be positioned within close proximity to its target such that the therapy produced by the cells is precisely targeted.

Thus, a desirable cell factory needs to have an immune barrier, while providing for diffusive transport of nutrients to the cell, respiratory byproducts from the surrounding area, and selected compounds to surrounding tissue. The immune barrier properties are required especially for use of non-host derived cell sources or designer deoxyribonucleic acid (DNA) manipulated cells.

As an exemplary application of bioreactors and cellular factories, electrical insulating coatings for implanted heart pacemakers and other electrically conductive medical devices may include therapeutic and cellular components (cells) such as anti-inflammatory or anti-thrombotic agents, which are produced in vivo for the prolonged use, thereby increasing the effectiveness of the device.

Encapsulated cell therapy systems hold promise for a range of cell-based delivery for long-term therapeutics that treat diabetes, renal failure, hemophilia, cardiovascular diseases, lysosomal storage diseases, Huntington's disease, ophthalmic disorders, chronic pain, musculoskeletal diseases, hormonal growth deficiencies, solid tumors, and central nervous system diseases such as amyotrophic lateral sclerosis (ALS or Lou Gehrig's disease) and Parkinson's disease. For example, encapsulated cells may enable the directed delivery of highly toxic chemotherapies to cancerous tumors, increasing the options of using chemotherapies, which were previously too toxic, in a localized and localizable fashion. Diabetes is one of the most significant areas of current research for the encapsulation of cells, specifically islet cells of the pancreas that produce insulin. Encapsulated cell therapy is being studied for use in gene therapies such as viral vector designer deoxyribonucleic acid (DNA) from endogenous harvested cells that are vector modified prior to implantation and then implanted.

In cell encapsulation, transplanted cells can be protected from immune rejection by an artificial, semi-permeable membrane such as alginate. Alginate gels have been used in biomedical applications to immobilize living cells or other biomaterials, maintaining good cell viability during long-term culture in the mild environment of the gel network. Conventional pharmaceutical-grade alginate, which is low in endotoxins and other impurities, is extracted from marine brown algae and produced by certain bacteria, for example, Azotobacter vinelandii.

Recently, medical researchers have encapsulated genetically engineered cells and therapeutic cells in immuno-isolating substances to deliver specific substances to targeted treatment areas such a brain tumors. Within tissue-engineering applications, immobilized cells or tissues may be able to serve as bio-artificial organs, while surrounding immuno-isolating substances function as a protection from physical stress and immunological reactions with the host. These cell bioreactors have the potential to excrete biopharmaceuticals and other therapeutic products, and are being clinically tested for the treatment of a variety of diseases like cancer and diabetes. In the case of brain tumors, encapsulated producer cells could be an in-vivo delivery system for specific proteins that target phenotypic features and micro-environmental factors, thereby interfering with tumor growth and differentiation.

In light of the forgoing discussion, targeted and controlled long-term delivery of therapeutic drugs, genes or cells, along with their enscapsulation material still need to be optimized with regard to biocompatibility, mechanical and chemical stability, suitable permeability, immune protection for cellular therapeutics, and the transfer of therapeutic material within a mammalian body.

Successful methods and systems for delivery of cellular therapies are needed to maintain viable transplanted or implanted cells that produce desirable compounds for extended treatment. Improved long-term delivery systems for therapeutic agents may be compliant to surrounding tissues and organs and avoid malapposition of medical devices. Ideally, an encapsulation material and delivery system for various types of pharmacological, gene, and cell therapies eliminate the need for immuno-modulatory protocols or immunosuppressive drugs, and permit the long-term de novo delivery of therapeutic products to either a localized area or overall life system.

B. Systems for Forming Bioreactors In Situ

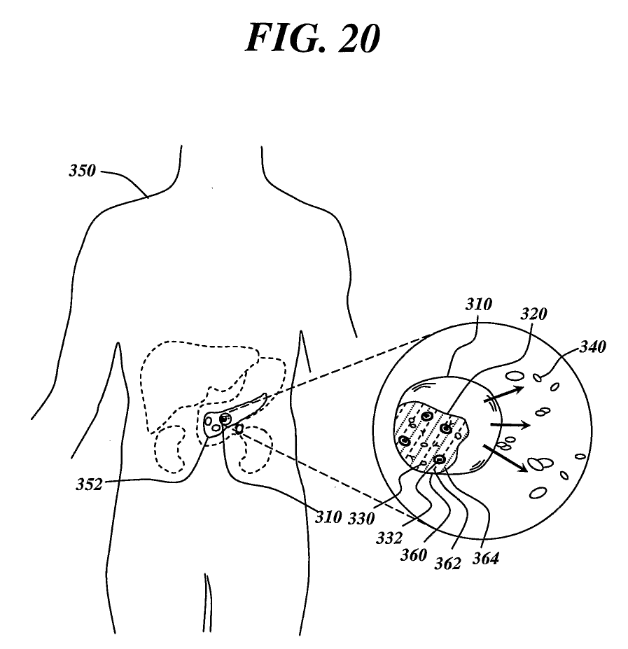

FIG. 20 illustrates an alginate bioreactor 310 for treating a mammalian body 350, in accordance with one embodiment of the present invention. A cutaway view of an exemplary in-situ formed alginate reactor 310 is shown in the inset. Alginate bioreactor 310 includes an alginate matrix 320 and one or more therapeutic components 330 or cellular components 332 dispersed within alginate matrix 320. A therapeutic agent 340 is eluted from alginate matrix 320 after alginate bioreactor 310 is formed within mammalian body 350. Alginate matrix 320 of alginate bioreactor 310 may be formed from an alginate solution 360 injected into a portion 352 of mammalian body 350 such as a pancreas. Alginate bioreactor 310 may be located in a portion of mammalian body 350 such as a heart, a liver, a pancreas, a kidney, an eyeball, a pericardial space, a cerebral spinal space, a periorganic space, an organ, a vessel, or a tissue.

In one embodiment, the invention provides localized delivery of one or more therapeutic agents 340 from therapeutic components 330 dispersed within alginate bioreactor 310 when alginate bioreactor 310 is formed within mammalian body 350 of a mammalian recipient. In another embodiment of the invention, one or more therapeutic agents 340 are delivered long-term from a matrix suitable for maintaining encapsulated cells and aggregates of viable cells from transplanted or implanted cells that produce such therapeutic agents. In yet another embodiment, one or more therapeutic agents 340 are delivered long-term from an alginate matrix 320 that may have one or more therapeutic components 330 and one or more cellular components 332 dispersed therein. When alginate matrix 320 is employed, therapeutic components 330 and cellular components 332 may be uniformly dispersed throughout alginate bioreactor 310, have a non-uniform profile with a higher concentration of therapeutic components 330 or cellular components 332 nearer the center, or have a non-uniform profile with a higher concentration of therapeutic components 330 and cellular components 332 closer to an outer surface of alginate bioreactor 310. In another example, therapeutic components 330 and cellular components 332 agglomerate or collect in regions within alginate bioreactor 310.

Alginate bioreactor 310 is formed from alginate solution 360 that is injected by an alginate injection system into portion 352 of mammalian body 350. Formed alginate bioreactor 310 includes an alginate matrix 320. A syringe, an adapter catheter, high-pressure jets, or other injection techniques may be used to inject alginate solution 360 into the desired location in mammalian body 350.

Alginate bioreactor 310 elutes and locally delivers one or more therapeutic agents 340 from therapeutic components 330 and cellular components 332 contained therein to treat medical conditions within mammalian body 350.

Alginate bioreactor 310 provides a mechanism for controlled, time-release characteristics of therapeutic agents 340 from any therapeutic components 330 and cellular components 332 within alginate matrix 320 of alginate bioreactor 310. Delivery of therapeutic agents 340 may occur over days, weeks, months and even years after formation of alginate bioreactor 310. With cellular components 332, therapeutic agents 340 may be continuously produced over the lifetime of the host. In one embodiment, the invention provides localized delivery of one or more therapeutic agents 340 from therapeutic components 330 dispersed within alginate bioreactor 310 when alginate bioreactor 310 is formed within mammalian body 350 of the mammalian recipient. In another embodiment, the invention provides long-term delivery of one or more therapeutic agents 340 via alginate matrix 320 that is suitable for maintaining encapsulated cells and aggregates of viable cells from transplanted or implanted cells that produce such therapeutic agents 340.

In one embodiment, alginate bioreactor 310 includes one or more therapeutic components 330 dispersed within alginate matrix 320, which controls the elution of therapeutic agent 340 from alginate bioreactor 310. Therapeutic component 330 includes, for example, an anti-coagulant, an anti-platelet drug, an anti-thrombotic drug, an anti-proliferant, an inhibitory agent, an anti-stenotic substance, heparin, a heparin peptide, an anti-cancer drug, an anti-inflammatant, nitroglycerin, L-arginine, an amino acid, a nutraceutical, an enzyme, a nitric oxide synthase, a diazeniumdiolate, a nitric oxide donor, rapamycin, a rapamycin analog, paclitaxel, a paclitaxel analog, a coumadin therapy, a lipase, a protein, insulin, bone morphogenetic protein, or a combination thereof. Therapeutic agents 340 released from alginate bioreactor 310 include, for example, therapeutic components 330 themselves or portions thereof.

In another embodiment, one or more cellular components 332 are dispersed within alginate matrix 320 of alginate bioreactor 310 to provide therapeutic agent 340. Alginate matrix 320 provides an immune barrier for cellular components 332 and controls the elution of therapeutic agents 340 from alginate bioreactor 310. Cellular component 332 includes, for example, endothelial cells, manipulated cells of designer deoxyribonucleic acid, host-derived cells from a host source, donor-derived cells from a donor source, pharmacologically viable cells, freeze-dried cells, or a combination thereof. Therapeutic components 330 along with cellular components 332 may elute one or more therapeutic agents 340 into surrounding tissue.

Exemplary alginate matrix 320 includes selected therapeutic components 330 and cellular components 332 that produce therapeutic agents 340 for elution from alginate matrix 320 of alginate bioreactor 310. When cellular components 332 are selected, alginate matrix 320 may serve as an immune barrier so that the immune system of the recipient does not recognize and destroy cellular component 332 contained within alginate matrix 320, or terminate the production of therapeutic agents 340. Meanwhile, alginate matrix 320 still allows for the metabolic transfer of nutrients, wastes, and therapeutic proteins and agents to pass through alginate matrix 320 into surrounding mammalian body 350. Therapeutic agents 340 are delivered in close proximity to the treatment site and released from alginate bioreactor 310. Alginate bioreactor 310 with therapeutic components 330 and cellular components 332 provides long-term expression of the therapeutic agents 340.

Therapeutic agents 340 from cellular components 332 include, for example, a residue, a byproduct, or natural excretion from the cells. One exemplary therapeutic agent 340 is nitric oxide.

Alginate bioreactor 310 having therapeutic components 330 or cellular components 332 may help prevent, for example, inflammation or rupture of tissue by eluting of one or more therapeutic agents 340. For example, eluted therapeutic agents 340 may reduce inflammation in the vicinity of alginate bioreactor 310 and the area of mammalian body 350 being treated.

Alginate bioreactor 310 may take the form of an indwelling filter for venous applications that incorporate cellular components 332 and elute therapeutic agents 340 such as streptokinases, kinases or other thrombolytic agents, coumadin materials or other blood thinning agents, nitrous oxide, and other agents.

Living cells or other biomaterials and therapeutic compounds can be immobilized in alginate matrix 320 such as an alginate gel. Cells immobilized in alginate gels maintain good viability during long-term culture, due in part to the mild environment of the gel network. Alginate gel provides a physically protective barrier for immobilized cells and tissue, and inhibits immunological reactions of the host. Alginate matrix 320 provides a location that is viable and productive for cellular components 332, since alginate matrix 320 allows the diffusion of nutrients to the cell, diffusion of respiratory byproducts to the surrounding area, and diffusion of selected therapeutic components 330 in an unaltered condition from alginate matrix 320. In some cases, alginate matrix 320 serves as an immune barrier while providing for diffusive transport for therapeutic and cellular materials. The immune barrier properties of alginate matrix 320 are particularly useful for non-host derived cell sources, or manipulated cells of designer deoxyribonucleic acid (DNA). Viral transfection of desirable DNA can occur outside mammalian body 350 into cellular components 332 that are encapsulated in situ, reducing the possibility of reaction to the viral vector itself, and allowing for more DNA to be transfected into alginate bioreactor 310.

One example of a cellular component 332 is endothelial cells that produce nitric oxide, a regulating molecule for smooth muscle cell quiescence and maintenance of vascular smooth muscle cells in the non-proliferative stage. A patient's own endothelial cells from, for example, microvascular adipose tissue, may be harvested and mixed with alginate solution 360, and formed along with alginate matrix 320 into alginate bioreactor 310. Upon implantation, the endothelial cells remain viable and locally produce nitric oxide to regulate and maintain the quiescent nature of smooth muscle cells, which can be a contributor to the production and recruitment of fibroblasts from the media and adventitia of arteries. With the continued long-term production of nitric oxide from the translocated endothelial cells, vascular patency may be maintained for a substantially longer period following bioreactor formation.

Long-term administration of at least one therapeutic agent 340 such as nitric oxide may be provided to portion 352 of mammalian body 350 that is diseased or traumatized. For example, disruption of the endothelial lining in a diseased portion of mammalian body 350 may result in the reduction of nitric oxide production, leading to the loss of regulation of the smooth muscle cells. Endothelial-derived nitric oxide is a naturally occurring regulation compound that can be produced by, for example, the endothelial cell lining of blood vessels. Endogenously produced nitric oxide molecules can regulate the proliferation of the vascular smooth muscle cells and maintain the cellular quiescence of smooth muscle cells within the vascular architecture. Nitric oxide is also critical to numerous biological processes, including vasodilation, neurotransmission, and macrophage-mediated microorganism and killing of tumors. Nitric oxide may be administrated in a chemically synthesized form as a nitric oxide donor, such as nitroglycerin dispersed within alginate matrix 320.

Since it is such a small molecule, nitric oxide is able to diffuse rapidly across cell membranes and, depending on the conditions, is able to diffuse distances of more than several hundred microns, as is demonstrated by its regulation of smooth muscle cells, vascular dilation, tissue compliance and physiological tone of the mammalian body. Nitric oxide can be produced within alginate matrix 320 and delivered directly to the mammalian body. For example, L-arginine, a naturally occurring amino acid, and other nutraceuticals are converted to nitric oxide within alginate matrix 320 by a group of enzymes such as nitric oxide synthases. These enzymes convert L-arginine into citrulline, producing nitric oxide in the process. In another example, nitric oxide is liberated from diazeniumdiolates, compounds that release nitric oxide into the blood stream and vascular walls.

Alginate matrix 320 may comprise a predetermined ratio of mannuronate alginate subunits 362 and guluronate alginate subunits 364.

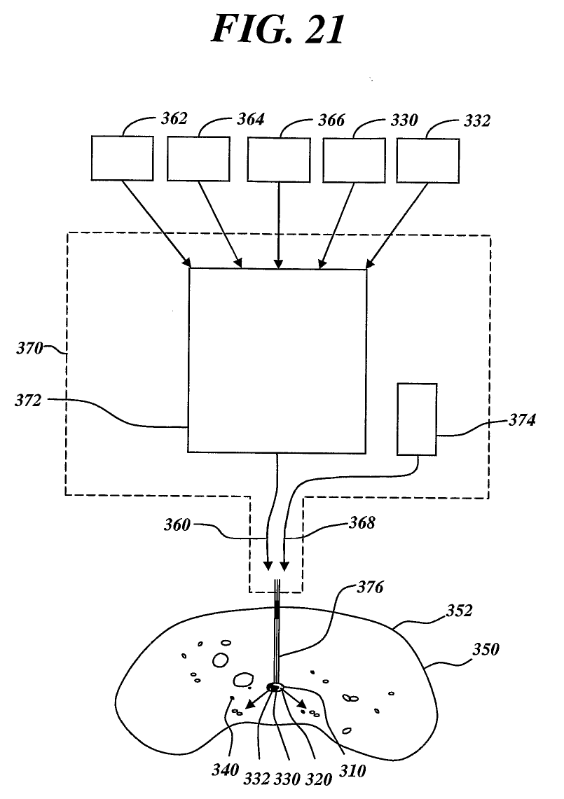

FIG. 21 illustrates a system for forming an alginate bioreactor 310 in a portion 352 of a mammalian body, in accordance with one embodiment of the present invention. An alginate bioreactor 310 is being formed within a portion 352 of mammalian body 350 such as a kidney. An alginate injection system 370 includes a first chamber 372, a second chamber 374, and an alginate solution injector 376, the latter being fluidly coupled to first chamber 372 and second chamber 374. An alginate solution 360 from-first chamber 372 is injected into portion 352 of the mammalian body with an alginate linking agent 368 from second chamber 374 to form alginate bioreactor 310.

Alginate bioreactor 310 within portion 352 of the mammalian body provides directed, localized, time-released delivery of therapeutic agents 340 from therapeutic components 330 and/or cellular components 332 dispersed within alginate bioreactor 310. In one embodiment, alginate bioreactor 310 with alginate matrix 320 encapsulates and maintains the viability of cellular components 332 and allows the expression of therapeutic agents 340 from the cells to pass through alginate matrix 320 and elute into surrounding targets such as organs, vessels, and other portions of the mammalian body.

A ratio of mannuronate alginate subunits 362 and guluronate alginate subunits 364 may be selected to provide a predetermined elution characteristic of alginate bioreactor 310. An alginate premix of mannuronate alginate subunits 62 and guluronate alginate subunits 364, an alginate solvent 366 such as alcohol or water, and one or more therapeutic components 330 and cellular components 332 are combined to form alginate solution 360 with the determined ratio of mannuronate alginate subunits 362 and guluronate alginate subunits 364. Alginate linking agent 368 may be added to alginate solution 360 or maintained separately until combined in the mammalian body. When alginate solution 360 and alginate linking agent 368 are injected into the mammalian body, the alginate cross-links, gels, and hardens to form alginate bioreactor 310. Cross-linking and polymerization of alginate solution 360 may occur in situ while at body temperature, or activated with exposure to ultraviolet light, infrared light, or thermal energy.

Alginate solution injector 376, such as a single-lumen syringe, may be used to inject the combined or separated alginate solution 360 and alginate linking agent 368 into the mammalian body. In cases where alginate solution 360 and alginate linking agent 368 remain separated until injected into the mammalian body, a double-lumen syringe may be used for local injection. Alternatively, endoscopic techniques using, for example, guidewires and a bioreactor formation catheter with one or more delivery lumens, inject alginate solution 360 and alginate linking agent 368 endoscopically into the mammalian body. Alternatively, a high-pressure injection nozzle or a pair of high-pressure injection nozzles injects alginate solution 360 and alginate linking agent 368 into the mammalian body.

The alginate bioreactor may include an alginate matrix and one or more therapeutic components and cellular components dispersed therein. Formation of the alginate bioreactor may occur in a clinical setting, so that donor-provided cells, for example, may be harvested from a host or donor mammalian body and combined into the alginate solution immediately prior to formation of the alginate bioreactor.

The alginate bioreactor is formed within a portion of the mammalian body to provide controlled, time-released delivery of therapeutic agents from therapeutic components and/or cellular components dispersed within the alginate bioreactor. In one embodiment, the alginate bioreactor with an alginate matrix encapsulates and maintains the viability of cellular components, and allows the expression of therapeutic agents from the cells to pass through the alginate matrix and elute into surrounding targets such as arterial tissues, vessels, organs, and periorganic spaces.

Desired therapeutic components and cellular components are selected along with the desired quantity. Selectable therapeutic components include, for example, an anti-coagulant, an anti-platelet drug, an anti-thrombotic drug, an anti-proliferant, an inhibitory agent, an anti-stenotic substance, heparin, a heparin peptide, an anti-cancer drug, an anti-inflammatant, nitroglycerin, L-arginine, an amino acid, a nutraceutical, an enzyme, a nitric oxide synthase, a diazeniumdiolate, a nitric oxide donor, rapamycin, a rapamycin analog, paclitaxel, a paclitaxel analog, a coumadin therapy, a lipase, a protein, insulin, bone morphogenetic protein, or a combination thereof. Selectable cellular components include, for example, endothelial cells, designer-DNA manipulated cells, host-derived cells from a host source, donor-derived cells from a donor source, pharmacologically viable cells, freeze-dried cells, and combinations thereof. The dose and constituency of added therapeutic and cellular components may be selected based on the desired treatment of the mammalian body and the desired elution rate of the therapeutic agents.

A ratio of mannuronate alginate subunits and guluronate alginate subunits may be determined to provide a predetermined elution characteristic of the alginate bioreactor, based on the desired elution characteristics of the therapeutic and cellular components. For example, the block length of mannuronate alginate subunits and the block length of guluronate alginate subunits are selected to achieve suitable strength and flexibility of the bioreactor, while providing controlled delivery of therapeutic and cellular components dispersed within the alginate matrix.

Prior to injection and formation of the alginate bioreactor, the alginate premix, monomers or polymers may be sterilized by passage through a selection of submicron filters, by exposure to radiation in the form of ionizing gamma or electron beams, or by other known methods of rendering a viscous solution sterile. The premix may be mixed in a suitable solvent prior to filtration and then dried, for example, by dialysis or spray drying.

An alginate solution including an alginate premix and an alginate solvent is mixed prior to forming the alginate bioreactor. In one example, the mannuronate alginate subunits, guluronate alginate subunits, and an alginate solvent such as alcohol or water are mixed to form the alginate solution with the determined ratio of mannuronate alginate subunits and guluronate alginate subunits. The concentration and viscosity of the alginate solution may be reduced with the addition of aqueous cellular or therapeutic components. In another example, the mannuronate alginate subunits, guluronate alginate subunits, alginate solvent, and the selected therapeutic or cellular components are combined to form the alginate solution with the determined ratio of mannuronate alginate subunits and guluronate alginate subunits. For example, endothelial cells are mixed into a formulation of alginate with appropriate mannuronate and guluronate components into an alginate solution, and the alginate solution used to form the alginate bioreactor. In another example, an alginate premix of mannuronate alginate subunits and guluronate alginate subunits, an alginate solvent such as alcohol or water, and one or more therapeutic components and cellular components are combined to form the alginate solution.

In an optional step, one or more viable cell components may be harvested from the host or a donor mammalian body and mixed into the alginate solution prior to formation of the alginate bioreactor in the mammalian body, as seen at block 84. The cellular component may be genetically manipulated prior to forming the alginate bioreactor. The harvested cells may be further cultured to increase their numbers or further filtered to obtain the desired quantity, quality and type of cells. The harvested viable cellular component, such as endogenous endothelial cells, is mixed into the alginate solution prior to injecting the alginate solution. In another example, freeze-dried cells are mixed into the alginate solution with, for example, an alcohol-based alginate solvent. The freeze-dried cells are reconstituted after the alginate bioreactor is formed within the mammalian body. In another example, cells from either a host or donor source are preserved with trehalose and freeze-dried, rendering the cells functional yet in a dehydrated state. Use of cells in a preserved fashion allows for mixing the alginate solution with the cells in advance or conjointly with the medical procedure. One skilled in the art can identify alternative cell-producing components that can be substituted for endothelial cells and provide therapeutic products from the alginate matrix.

An alginate linking agent is provided, and the alginate solution and the alginate linking agent are injected into a portion of the mammalian body with an alginate injection system. The alginate bioreactor is formed by injecting an alginate solution and an alginate linking agent into the portion of the mammalian body, and hardening the alginate solution to form the alginate bioreactor. The added alginate linking agent comprises, for example, divalent calcium, divalent barium, divalent strontium, divalent magnesium, a divalent cation, or a source of calcium such as a calcium salt.

In one example, the alginate linking agent is added to the alginate solution immediately prior to injecting the alginate solution into the portion of the mammalian body, due to rapid gelling and setting of the alginate matrix. In another example, the alginate linking agent is added to the alginate solution after injecting the alginate solution into the portion of the mammalian body. In another example, the alginate linking agent is co-injected into a portion of the mammalian body to form the bioreactor. In another example, the alginate linking agent is deposited in the portion of the mammalian body prior to injecting the alginate solution. In another example, the alginate linking agent is injected into the mammalian body and combined with alginate solution injected from a separate source. In another example, the alginate linking agent is deposited, applied, diffused, or otherwise transferred to the portion of the mammalian body prior to injecting the alginate solution. As the alginate solution is injected, the alginate solution coagulates within the portion of the mammalian body to form the alginate bioreactor.

The alginate solution is injected into a portion of the mammalian body, where the alginate solution cross-links, gels, and hardens to form the alginate bioreactor. The alginate bioreactor includes an alginate matrix and one or more therapeutic and cellular components. The amount of alginate solution injected into the mammalian body is related to the size, quantity and density of the formed bioreactor.

In one example, the alginate solution is injected into the portion of the mammalian body with a syringe having at least one lumen. In another example, alginate solution is injected through a bioreactor formation catheter into a sidewall of a vessel, heart, or other endoscopically accessible portion of the mammalian body. The bioreactor formation catheter is positioned, for example, by advancing the distal end of the bioreactor formation catheter over a catheter guidewire to a treatment site in the vessel, a medical procedure as is known in the art. In another example, the alginate solution is injected into the portion of the mammalian body with a high-pressure jet.

Once the alginate bioreactor is formed, one or more therapeutic agents may be eluted from therapeutic or cellular components that are dispersed within the alginate bioreactor. Exemplary eluted therapeutic agents from an alginate bioreactor having therapeutic or cellular components may include any of the therapeutic agents described above in Section II. In one example, the eluted therapeutic agent comprises nitric oxide from entrained endothelial cells to regulate the proliferation of smooth muscle cells in the mammalian body near the formed alginate bioreactor. In another example, the cellular component is reconstituted in the alginate bioreactor, and the therapeutic agent is released from the reconstituted cellular component.

When a cellular component is employed, an alginate bioreactor is formed by a cellularized alginate solution at a location in the mammalian body where the cellular component is able to produce and elute a therapeutic agent while reconstituting itself for continued production of the agent. The immune barrier of the alginate matrix protects the cellular components while the alginate bioreactor controls the elution of the therapeutic agent from therapeutic and cellular components within the matrix.

The disclosure set forth above may encompass one or more distinct inventions, with independent utility. Each of these inventions has been disclosed in its preferred form(s). These preferred forms, including the specific embodiments thereof as disclosed and illustrated herein, are not intended to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A coated stent, comprising:
a stent latticework; and
an alginate coating disposed on the stent latticework.

2-6. (canceled)

7. The coated stent of claim 1 further comprising:
a therapeutic component dispersed within the alginate coating, wherein the therapeutic component acts as source of a therapeutic agent, and wherein the alginate coating controls elution of the therapeutic agent from the alginate coating.

8. The coated stent of claim 7, wherein the therapeutic component is selected from the group consisting of an anti-coagulant, an anti-platelet drug, an anti-thrombotic drug, an anti-proliferant, an inhibitory agent, an anti-stenotic substance, heparin, a heparin peptide, an anti-cancer drug, an anti-inflammatant, nitroglycerin, L-arginine, an amino acid, a nutraceutical, an enzyme, a nitric oxide synthase, a diazeniumdiolate, matrix metalloproteinase, a nitric oxide donor, rapamycin, a rapamycin analog, paclitaxel, a paclitaxel analog, a coumadin therapy, a lipase, and a combination thereof.

9. The coated stent of claim 1 further comprising:
a cellular component dispersed within the alginate coating, wherein the cellular component controllably releases a therapeutic agent when the coated stent is deployed within a vessel of a mammalian body.

10. (canceled)

11. The coated stent of claim 9, wherein the released therapeutic agent includes nitric oxide.

12. (canceled)

13. A method of treating a vessel in a mammalian body, the method comprising:
providing a stent latticework;
coating the stent latticework with an alginate solution to form a coated stent having an alginate coating disposed on the stent latticework;
positioning the coated stent within the vessel;
deploying the coated stent; and
eluting a therapeutic agent from the alginate coating.

14-16. (canceled)

17. The method of claim 13, wherein the alginate coating includes one of a therapeutic component or a cellular component.

18-19. (canceled)

20. The method of claim 13 further comprising:
determining a ratio of mannuronate alginate subunits and guluronate alginate subunits to provide a predetermined elution characteristic of the alginate coating;
mixing mannuronate alginate subunits, guluronate alginate subunits, an alginate solvent, and one of a therapeutic component or a cellular component to form an alginate solution with the determined ratio of mannuronate alginate subunits and guluronate alginate subunits;
adding an alginate linking agent to the alginate solution; and
coating the stent latticework with the alginate solution.

21. (canceled)

22. The method of claim 13 further comprising:
selecting at least one of a therapeutic component and a cellular component; and
mixing the selected at least one component into the alginate solution prior to coating the stent latticework.

23. The method of claim 13 further comprising:
harvesting a viable cellular component from the mammalian body; and
mixing the harvested viable cellular component into the alginate solution prior to coating the stent latticework.

24-25. (canceled)

26. An alginate coating for an implantable medical device, the alginate coating comprising:
an alginate matrix; and
at least one of a therapeutic component and a cellular component dispersed within the alginate matrix.

27. (canceled)

28. An alginate implant for treating a vessel in a mammalian body, the alginate implant comprising:
an alginate matrix in contact with an endoluminal wall of the vessel; and
a central lumen axially extending through the alginate matrix.

29-38. (canceled)

39. The alginate implant of claim 28, wherein the implant is configured as at least one of a stent and a cap for vulnerable plaque.

40. A method of treating a vessel in a mammalian body, the method comprising:
forming an alginate implant within the vessel, the alginate implant in contact with an endoluminal wall of the vessel and having a central lumen axially extending through the alginate implant; and
eluting a therapeutic agent from one of a therapeutic component or a cellular component dispersed within the alginate implant.

41-48. (canceled)

49. The method of claim 40 further comprising:
determining a ratio of mannuronate alginate subunits and guluronate alginate subunits to provide a predetermined elution characteristic of the alginate implant;
combining mannuronate alginate subunits, guluronate alginate subunits, the alginate solvent, and the therapeutic component or the cellular component to form the alginate solution with the determined ratio of mannuronate alginate subunits and guluronate alginate subunits;
adding an alginate linking agent into the alcinate solution: and
injecting the alginate solution into a portion of the vessel with an implant formation catheter.

50-52. (canceled)

53. A system for forming an alginate implant in a mammalian body, the system comprising:
an implant formation catheter having a catheter body;
a formation balloon attached to the catheter body near a distal end of the catheter body; and
an alginate-delivery lumen within the catheter body, wherein an alginate implant is formed from an alginate solution injected through the alginate-delivery lumen into a cavity between the formation balloon and an endoluminal wall of the vessel when the formation balloon is inflated.

54-58. (canceled)

59. A method of forming an alginate implant in a vessel of a mammalian body, the method comprising:
positioning an implant formation catheter in the vessel, the implant formation catheter having a catheter body;
inflating a distal occlusion balloon attached to the catheter body near a distal end of the catheter body;
inflating a proximal occlusion balloon attached to the catheter body proximal to the distal balloon;
inflating a medial formation balloon attached to the catheter body between the distal occlusion balloon and the proximal occlusion balloon;
injecting an alginate solution through an alginate-delivery lumen into a cavity formed between the inflated distal occlusion balloon, the inflated proximal occlusion balloon, the inflated medial formation balloon, and an endoluminal wall of the vessel; and
hardening the alginate solution to form the alginate implant.

60-62. (canceled)

63. A method of forming an alginate implant in a vessel of a mammalian body, the method comprising:
positioning an implant formation catheter at a first location in the vessel, the implant formation catheter having a catheter body;
inflating an angioplasty balloon attached to the catheter body near a distal end of the catheter body, the angioplasty balloon having an alginate linking agent disposed on a surface of the angioplasty balloon;
depositing the alginate linking agent on an endoluminal wall of the vessel;
deflating the angioplasty balloon;
repositioning the implant formation catheter at a second location in the vessel, the second location in the vessel distal to the first location in the vessel;
re-inflating the angioplasty balloon;
inflating a formation balloon attached to the catheter body proximal to the angioplasty balloon;
injecting an alginate solution through an alginate-delivery lumen into a cavity formed between the formation balloon and an endoluminal wall of the vessel; and
hardening the alginate solution to form the alginate implant, wherein the alginate solution is hardened by the alginate linking agent deposited on the endoluminal wall of the vessel.

64-67. (canceled)

68. A method of forming an alginate implant in a vessel of a mammalian body, the method comprising:
inserting an implant formation catheter into the vessel, the implant formation catheter having at least one formation balloon;
injecting an alginate solution into a cavity formed between the formation balloon and an endoluminal wall of the vessel when the formation balloon is inflated;
hardening the alginate solution to form the alginate implant; and
withdrawing the implant formation catheter from the vessel, wherein the formed alginate implant is in contact with the endoluminal wall of the vessel and includes a central lumen axially extending through the alginate implant.

69. An alginate bioreactor for treating a mammalian body, the alginate bioreactor comprising:
an alginate matrix; and
one of a therapeutic component or a cellular component dispersed within the alginate matrix, wherein a therapeutic agent is eluted from the alginate matrix after the alginate bioreactor is formed within the body.

70. (canceled)

71. The alginate bioreactor of claim 69, wherein the alginate bioreactor is formed in a portion of the mammalian body, the portion of the mammalian body selected from the group consisting of a heart, a liver, a pancreas, a kidney, an eyeball, a pericardial space, a cerebral spinal space, a periorganic space, an organ, a vessel, and a tissue.

72-76. (canceled)

77. The alginate bioreactor of claim 69, wherein the eluted therapeutic agent is selected from the group consisting of vascular endothelial growth factor, a biological anti-inflammatory agent, vitamin C, acetylsalicylic acid, a lipid lowering compound, a high-density lipoprotein cholesterol, a streptokinase, a kinase, a thrombolytic agent, an anti-thrombotic agent, a blood-thinning agent, a coumadin material, an anti-cancer agent, an angiogenic agent, an anti-angiogenic agent, an anti-rejection agent, a hormone, a therapeutic component, a cellular component, and a combination thereof.

78. A method of treating a medical condition in a mammalian body, the method comprising:
forming an alginate bioreactor within a portion of the mammalian body, the alginate bioreactor including an alginate matrix; and
eluting a therapeutic agent from one of a therapeutic component or a cellular component dispersed within the alginate bioreactor.

79-97. (canceled)
